(19) United States
(12) Patent Application Publication
(10) Pub. No.: US 2008/0281421 A1
(43) Pub. Date: Nov. 13, 2008

(54) Wound Closure System and Methods

(76) Inventors: Frederick Cahn, La Jolla, CA (US); Moreno White, San Diego, CA (US)

Correspondence Address: KALOW & SPRINGUT LLP, 488 MADISON AVENUE, 19TH FLOOR, NEW YORK, NY, 10022, US

(21) Appl. No.: 11/597,398

(22) PCT Filed: May 10, 2005
(86) PCT No.: PCT/US2005/016321

§ 371 Date: Feb. 4, 2008

Related U.S. Application Data
(60) Provisional application No. 60/573,877, filed on May 24, 2004.

(51) Int. Cl. A61F 2/10

(52) U.S. Cl. 623/15.12

(57) ABSTRACT

Wound closure systems and methods are provided, containing a porous layer comprising a collagen material; a substantially non-porous synthetic layer contacting the porous layer, the porous layer and substantially non-porous layer capable of providing wound closure; and a transcutaneous component contacting the porous layer and the substantially non-porous synthetic layer. In various embodiments, the transcutaneous component is capable of receiving a cannula, glucose sensor, electrode, prosthesis, chest tube, medical instrument or bone, muscle, blood vessels, nerve, organ or combination thereof.

### BACKGROUND OF THE INVENTION

For centuries, mankind has envisioned directly attaching a transcutaneous component, such as for example, an artificial limb through the skin directly into the skeleton. Direct skeletal loading has the obvious difficulty of designing a suitable implant that is biocompatible, distributes load efficaciously over the skeletal section to which it is attached, and allows for passage through the skin so that an artificial limb can be attached. While early studies have substantiated the dramatic impact direct transcutaneous skeletal attachment devices can afford amputees, the current system has many unresolved problems. Unfortunately, there is no true biologic integration of the skin to the prosthesis. Without a biologic interface, bacteria can migrate along the surface between the skin and the device and inevitably, infections occur in time frames from several weeks to several years.

To date, aside from conventional skin grafting, there has been only one method of closing a skin wound that cannot close spontaneously. This method involves using artificial skin to provide wound closure. Artificial skin has a porous layer containing collagen and glycosaminoglycan (collagen-GAG), and a layer of silicone. Artificial skin provides immediate closure of the wound, which reduces local symptoms of inflammation, fluid loss, and later provides permanent and functional skin by reestablishing vascularization and regeneration of dermal and epidermal skin layers. Artificial skin also reduces the incidence of wound fibrosis, scars and/or contraction.

To provide wound closure, artificial skin is applied to the wound and allows the re-growth of neodermal tissue in the porous layer. After the neodermis is fully developed, which usually takes about 2 to 3 weeks, the silicone temporary layer is removed from the neodermis and an ultrathin “epidermal” autograft is applied to the neodermis. After the graft is applied, the wound closes and is fully healed after about one week.

Although artificial skin has been used to provide wound closure, it does not provide a means to close a wound created by a transcutaneous component. Thus, there is a need to develop new wound closure systems to support transcutaneous components such as for example, prosthetic devices, implants, cannulas, or other devices.

### SUMMARY OF THE INVENTION

In various embodiments of the present invention, a wound closure system is provided comprising one or more layers of artificial skin that supports a transcutaneous components, such as for example, prosthetic devices, implants, cannulas, or other devices.

In one embodiment, a wound closure system is provided, comprising: a porous layer which comprises a collagen material; a substantially non-porous synthetic layer contacting the porous layer, the porous layer and substantially non-porous layer capable of providing wound closure; and a transcutaneous component contacting the porous layer and the substantially non-porous synthetic layer. The transcutaneous component may have a porous portion that allows tissue ingrowth or artificial skin integration.

In another embodiment, a wound closure system is provided, comprising: a porous layer comprising a collagen material and a substantially non-porous synthetic layer contacting the porous layer, each layer capable of receiving a transcutaneous component, the porous layer and substantially non-porous layer capable of providing wound closure by allowing growth of neodermal tissue and an anchoring material disposed within the porous layer.

In still yet another embodiment, a wound closure system is provided, comprising: a porous layer and a substantially non-porous synthetic layer contacting the porous layer, each layer capable of being received or receiving a transcutaneous component, the porous layer comprising biodegradable collagen-glycosaminoglycan, the porous layer and substantially non-porous layer capable of providing wound closure by allowing growth of neodermal tissue, and a non-degradable anchoring material disposed within the porous layer.

In one preferred embodiment, a wound closure system is provided, comprising: a porous layer which comprises a collagen material; a substantially non-porous synthetic layer contacting the porous layer, the substantially non-porous synthetic layer comprising removable silicon and a permanent membrane, the porous layer and substantially non-porous layer capable of providing wound closure; and a transcutaneous component contacting the porous layer and the substantially non-porous synthetic layer.

In another preferred embodiment, a wound closure kit is provided, comprising: a porous layer comprising a collagen material capable of providing wound closure by allowing growth of neodermal tissue; and a substantially non-porous synthetic layer contacting the porous layer, the porous layer and substantially non-porous layer capable of receiving a pylon.

In yet another preferred embodiment, a method for providing wound closure surrounding a transcutaneous component is provided, comprising: applying a wound closure system to a wound, the wound closure system comprising: a porous layer comprising a collagen material that allows growth of neodermal tissue; a substantially non-porous synthetic layer contacting the porous layer; and a transcutaneous component surrounded by the porous layer and the substantially non-porous synthetic layer.

In yet another preferred embodiment, a transcutaneous infection (foreign body) barrier system is provided that reduces the risk of infection or foreign body entry into the wound closure system, comprising: a porous layer comprising a collagen material; a substantially non-porous synthetic layer contacting the porous layer, the porous layer and substantially non-porous layer capable of promoting wound closure; and a transcutaneous component contacting the porous layer and the substantially non-porous synthetic layer, the transcutaneous component having an integral subcomponent which allows physical incorporation of the porous and/or the non-porous layer into the transcutaneous component.

For a better understanding of various embodiments, reference is made to the following description taken in conjunction with the examples, the scope of which is set forth in the appended claims.

### BRIEF DESCRIPTION OF THE FIGURES

Preferred embodiments have been chosen for purposes of illustration and description, but are not intended in any way to restrict the scope of the claims. Preferred embodiments are shown in the accompanying figures, wherein:

FIG. 1 illustrates an embodiment of the wound closure system. In this embodiment, the wound closure system includes a crosslinked collagen-GAG biodegradable porous matrix, a silicone temporary layer, a permanent non-degradable impermeable biocompatible membrane contiguous with the silicone temporary layer, a permanent porous non-degradable biocompatible dermal anchor that is embedded in the collagen-GAG layer, and a permanent non-degradable sleeve which provides mechanical interfaces with the transcutaneous component, e.g. pylon or catheter, etc. The transcutaneous component or pylon surface may have a porous surface, which allows tissue ingrowth or artificial skin integration.

FIG. 2 illustrates typical composite reinforcement architectures for use in the transcutaneous component of the wound closure system.

FIG. 3 illustrates plots of typical glass-epoxy laminate properties as a function of lay-up architecture.

FIG. 4 illustrates comparison of composite reinforcing fibers specific strength and stiffness.

FIG. 5 illustrates stiffness, strength, and toughness comparisons for typical polymer matrices.

FIG. 6 is a schematic of a preferred embodiment of the wound closure system, which comprises lower extremity prosthesis. In this embodiment, the wound closure system includes a pylon comprising two sections: 1) proximal pylon, which is bonded with an adhesive or preferably, osseointegrated with the host bone and 2) the distal pylon, which incorporates the skin wound closure system. The two sections would be connected with a suitable structural connection such as a trunion or threaded joint.

### DETAILED DESCRIPTION OF THE INVENTION

Various embodiments will now be described. These embodiments are presented to aid in an understanding of the claims and are not intended to, and should not be construed to, limit the claims in any way. All alternatives, modifications and equivalents that may become obvious to those of ordinary skill on reading the disclosure are included within the spirit and scope of the claims.

### Terminology

Unless stated otherwise, the following terms and phrases have the meanings provided below:

### Tissue

An aggregation of similarly specialized cells united in the performance of a particular function.

### Skin

The outer integument or covering of the body, consisting of the dermis and the epidermis and resting upon the subcutaneous tissues.

### Wound

An injury or damage, usually restricted to those caused by physical means with disruption of the normal continuity of structures. Called also injury and trauma.

### Full-Thickness Skin Wound

A skin wound with the loss of epidermis, and all of the dermis or at least the depth of dermis that includes most or all sources of epidermal cells from epidermal adnexae (glands and follicles).

### Open Wound

A wound that communicates with the atmosphere by direct exposure.

### Clean Surgical Skin Wound

A full or partial thickness skin wound that is created by surgical excision or incision and that is free of necrotic tissue, without significant bleeding, and without significant microbial contamination.

### Wound Inflammation

A localized protective response elicited by injury or destruction of tissues, which serves to destroy, dilute, or wall off (sequester) both the injurious agent and the injured tissue. It is characterized in the acute form by the classical signs of pain (dolor), heat (calor) redness (rubor), swelling (tumor), and loss function (functio laesa). Histologically, it involves a complex series of events, including dilation of arterioles, capillaries, and venules, with increased permeability and blood flow; exudation of fluids, including plasma proteins; and leukocytic migration into the inflammatory focus.

### Wound Contraction

The shrinkage and spontaneous closure of open skin wounds.

### Wound Contracture

A condition of fixed high resistance to passive stretch of muscle, skin or joints resulting from fibrosis and scarring of the skin or the tissues supporting the muscles or the joints, or both.

### Granulation Tissue

The newly formed vascular tissue normally produced in the healing of wounds of soft tissue and ultimately forming the scar; it consists of small, translucent, red nodular masses or granulations that have a velvety appearance.

### Scar

Fibrous tissue replacing normal tissues destroyed by injury or disease.

### Wound Closure

The provision of an epithelial cover over a wound. It can be accomplished by approximating wound edges, performing a skin (auto)graft, or allowing spontaneous healing from the edges.

### Heal

To restore wounded parts or to make healthy.

### Healing

The restoration of integrity to injured tissue.

### Healing by First Intention

Healing in which union or restoration of continuity occurs directly without intervention of granulations.

### Healing by Second Intention

Union by closure of a wound with granulations, which form from the base and both sides toward the surface of the wound.

### Tissue Regeneration

Healing in which lost tissue is replaced by proliferation of cells, which reconstruct the normal architecture.

### Tissue Repair

Healing in which lost tissue is replaced by fibrous scar, which is produced from granulation tissue.

### Skin Replacement Surgery

Surgery that permanently replaces lost skin with healthy skin.

### Biomaterial

Any substance (other than a drug), synthetic or natural, that can be used as a system or part of a system that treats, augments, or replaces any tissue, organ, or function of the body.

### Graft

Any tissue or organ for implantation or transplantation.

### Autograft

A graft of tissue derived from another site in or on the body of the organism receiving it.

### Full Thickness Skin Autograft

A skin autograft consisting of the epidermis and the full thickness of the dermis.

### Split Thickness Skin Autograft

A skin autograft consisting of the epidermis and a portion of the dermis.

### Epidermal Autograft

An autograft consisting primarily of epidermal tissue, including keratinocyte stem cells, but with little dermal tissue.

### Engraftment

Incorporation of grafted tissue into the body of the host.

### Dermal Tissue Engraftment

Engraftment of dermal tissue resulting in reestablishment of vascular connections with cellular and extracellular matrix remodeling in the dermis.

### Epidermal Tissue Engraftment

Engraftment of an epidermal autograft by a process of epidermal tissue regeneration resulting in a confluent epidermis and permanent wound closure. (Epidermal appendages such as hair are not regenerated.)

### Wound Closure Immediate Physiological Response

An immediate restoration of some of the physiological functions of skin that is demonstrated by an immediate reduction in wound inflammation, pain, and fluid loss. Granulation tissue is not formed and wound contraction does not occur. In the case of a large wound, the open wound systemic physiological response is also reduced.

The headings below are not meant to limit the disclosure in any way; embodiments under any one heading may be used in conjunction with embodiments under any other heading.

### Wound Closure

In one embodiment, a wound closure system is provided, comprising: a porous layer which comprises a collagen material; a substantially non-porous synthetic layer contacting the porous layer, the porous layer and substantially non-porous layer capable of providing wound closure; and a transcutaneous component contacting or incorporating the porous layer and the substantially non-porous synthetic layer. In various embodiments, the wound closure system provides a transcutaneous infection barrier.

As used herein, “wound closure” is an art-recognized term and includes a surgical procedure for closing a clean surgical skin wound that can be accomplished by approximating wound edges, performing a skin autograft, or allowing spontaneous healing from the edges.

As used herein, a primary wound closure is a wound closure that provides healing by first intention. Primary wound closure includes, but is not limited to, a wound closure immediate physiological response. Primary wound closure may be accomplished with skin grafts to eventually provide an epithelial cover over the wound. Primary wound closure may also be accomplished by the artificial skin system, using a two-step procedure that is known in the art. Artificial skin provides a wound closure immediate physiological response followed by engraftment of the porous layer that creates new vascularized tissue called “neodermis.”

In one preferred embodiment, the wound closure system achieves primary closure or healing by the first intention.

### Porous Layer

The wound closure system includes a porous layer that mimics the dermis of the skin and is capable of receiving a transcutaneous component. Typically, the porous layer contains a hole, adapter or optionally a sleeve to receive the transcutaneous component and provides a snug fit around the transcutaneous component. The porous layer contacts or touches the substantially non-porous synthetic layer and may also contact the permanent membrane, the sleeve, dermal tissue, porous surface of the transcutaneous component or combinations thereof. Preferably, the layers are coated on one another.

By “porous layer” is meant that one or more layers are permeable to cellular elements that allow engraftment, vascularization, dermal remodeling and/or nutrients to the dermis. For example, the porous layer provides a scaffold for ingrowth of fibroblasts and vasculature, and allows regeneration of a permanent, autologous dermal tissue. In various embodiments, the porous layer is biodegradable and remodeled over a period of, for example, 1 or 2 month as the neodermal tissue regenerates. In various embodiments, the porous layer has pore sizes ranging from about 50 microns to about 350 microns. In various embodiments, the porous layer comprises a collagen material or any other material that allows growth of the neodermis as opposed to scar tissue. As used herein collagen material includes material that is tough, and fibrous. Collagen may be chemically synthesized and/or obtained from natural sources such as skin, tendons, bones, cartilage, and other connective tissues. The collagen may be chemically synthesized by methods known in the art, and may be crosslinked by methods known in the art. For example, in various embodiments, the porous layer comprises bovine hide or tendon collagen crosslinked with chondroitin-6 sulfate (collagen-glycosaminoglycan). The collagen material is biocompatible, e.g., it has a reduced tendency to generate the immune or inflammatory response. In various embodiments, the collagen material is remodelable by normal physiological mechanisms in wound closure. In various embodiments, the collagen material is biodegradable and not permanent, the biodegradable collagen material may break down by natural biological processes such as, for example, the growth of the neodermis.

In various embodiments, the porous layer contains an antimicrobial agent, growth factors (such as to grow new blood vessels), or growth inhibitors or combinations thereof. Some examples of antibiotics suitable for use, include, but are not limited to streptomycin, tetracycline, penicillin, vancomycin, clindamycin, erythromycin, polymyxin B, bacitracin, ciprofloxacin, rifampin, gentamicin, cefazolin, oxacillin, silver, silversulfadiazine and ampicillin, minocycline or combinations thereof. Some examples of growth factors suitable for use, include, but are not limited to keratinocyte growth factor, fibroblast growth factor, and the like.

### Anchoring Material

In various embodiments, the porous layer comprises one or more non-degradable or permanent anchoring material disposed within the porous layer. The anchoring material is capable of receiving the transcutaneous component or if a sleeve is employed, contacts the sleeve. The anchoring material absorbs the mechanical stress between the transcutaneous component and the skin. Suitable anchoring material for use includes, but is not limited to, silicone, polymers such as for examples, PTFE, nylon, Dacron, polyacrylate esters, polyurethane, polyetheretherketone (PEEK), polyaryletherketone, metal, such as for example, titanium, steel, stainless steel, noble metals, such as for example, platinum, palladium, gold, rhodium, or non-metals, such as for example, carbon, carbon fiber or boron or combinations thereof. In various embodiments, the anchoring material is porous and permanent and contains an inner region and an outer region. The inner region is adjacent to the transcutaneous component and is typically less flexible or more stiff than the outer region to minimize mechanical strain underneath the epidermal to device junction. In various embodiments, the anchoring material is a lens-shaped dermal anchor, however, the present invention, is not limited to any one particular shape. In various embodiments, the anchoring material may contain an antimicrobial agent, growth factors (such as to grow new blood vessels), or growth inhibitors or combinations thereof.

### Substantially Non-Porous Synthetic Layer

A substantially non-porous synthetic layer contacts or touches the porous layer and is designed to mimic the epidermis. Typically, the substantially non-porous synthetic layer is taped or sutured to the wound edges when the device is inserted. The substantially non-porous synthetic layer is capable of receiving the transcutaneous component. In various embodiments, the non-porous synthetic layer contains a hole, adapter or optionally a sleeve to receive the transcutaneous component and provides a snug fit around the transcutaneous component. The substantially non-porous synthetic layer may also contact the permanent membrane, dermal tissue, or combinations thereof. In various embodiments, the substantially non-porous synthetic layer is contiguous with the porous layer and may be removable. As used herein, “substantially non-porous synthetic layer” includes, but is not limited to, one or more layers that have been produced by chemical synthesis that have substantially no pores that allow contaminants into the porous layer. The substantially non-porous layer limits moisture transmission, bacteria, viruses, toxins, etc. The substantially non-porous layer adheres to the collagen material. In various embodiments, the non-porous synthetic layer has sufficient tear strength, and handling characteristics. In one embodiment, the substantially non-porous synthetic layer provides a moisture flux of from about 0.1 to about 1 mg/cm²/hr, which is the moisture flux of normal skin. In various embodiments, the substantially non-porous synthetic layer comprises silicone, polymers such as for examples, PTFE, nylon, Dacron, polyacrylate esters, polyurethane, polyacrylate esters like polyester, polyurethane, polybutylene, polypropylene, carbon, carbon fiber or combinations thereof. In various embodiments, the substantially non-porous synthetic layer is removable and comprises biocompatible silicone.

In various embodiments, the substantially non-porous synthetic layer may contain an antimicrobial agent, growth factors (such as to grow new blood vessels), or growth inhibitors or combinations thereof.

### Permanent Impermeable Membrane

In various embodiments, one or more permanent impermeable membranes are optionally disposed within the substantially non-porous synthetic layer and contacts the transcutaneous component. The permanent membrane is designed to be permanent and non-degradable and may also contact the sleeve, dermal tissue, porous layer or combinations thereof. In various embodiments, the sleeve may be bonded to the transcutaneous component or integral to the structural transcutaneous component and allows tissue ingrowth of skin. By “non-degradable” is meant that the permanent membrane cannot be substantially broken down by natural biological processes, such as for example, the growth of the epidermis. By “impermeable” is meant that the membrane is not substantially permeable to bacteria viruses, and toxins, and has a controlled moisture permeability.

In various embodiments, the permanent impermeable membrane comprises metal, such as for example, aluminum, titanium, zirconium, cobalt, chrome, steel, stainless steel, noble metals, such as for example, platinum, palladium, gold, rhodium, or non-metals, such as for example, carbon, carbon fiber, ceramic, glass, silicone, polymers such as for examples, PTFE, nylon, Dacron, polyacrylate esters like polyester, polyurethane, polybutylene, polypropylene, polyetheretherketone, polyaryletherketone, or combinations thereof. In various embodiments, the permanent impermeable membrane is biocompatible, impact resistant, and damage tolerant. The permanent impermeable membrane may be reinforced with supporting material depending on the size and shape of the transcutaneous component and adjusts to different conditions including mechanical strain.

In various embodiments, the permanent impermeable membrane provides a junction between the non-porous layer (and later the epidermis after regeneration of the skin) and the transcutaneous component, for example, the prosthetic device. The permanent impermeable membrane may or may not be flexible. If the permanent impermeable membrane is flexible, for example, the membrane will be compliant and match the flexation of the adjacent and underlying tissue when stress is applied. In various embodiments, the permanent impermeable membrane is disc shaped, however, the present invention is not limited to any one particular shape.

### Transcutaneous Component

The wound closure system employs one or more transcutaneous components that contact or touch the one or more substantially non-porous synthetic layer, and the one or more porous layer. In various embodiments, the transcutaneous component is surrounded by the substantially non-porous synthetic layer and the porous layer. In various embodiments, the transcutaneous component contacts at least one of the permanent impermeable membranes, anchoring material, sleeve or combination thereof.

By “transcutaneous” is meant that the component passes from the outside environment through the epidermis and completely or partially through the dermis. In various embodiments, the transcutaneous component passes through the skin and contacts muscle, bone, blood vessels, nerve, organ and other tissue that is typically covered by the epidermis and/or dermis. The transcutaneous component includes, but is not limited to, hollow members, solid members or combinations thereof. In various embodiments, the transcutaneous component is biocompatible, load bearing, impact resistant, and/or damage tolerant. In various embodiments, the transcutaneous component can be connected to a catheter, IV port, cannula, glucose sensor, electrode, prosthesis, chest tube, or other medical or surgical instrument, bone, muscle, blood vessels, nerve, organ or combinations thereof. In the most preferred embodiment, the transcutaneous component is a pylon, which can be a hollow member or a solid member.

In various embodiments, the transcutaneous component comprises one or more metals, such as for example, aluminum, titanium, zirconium, cobalt, chrome, steel, stainless steel, noble metals, such as for example, platinum, palladium, gold, rhodium, or combinations thereof, or fibers or polymers reinforced with, for example, boron or titanium. In various embodiments, the transcutaneous component comprises reinforced fibers, either continuous or discontinuous, that are capable of carrying a significant load, such as for example, the weight of a human body. In various embodiments, the transcutaneous component comprises non-metals, such as for example, carbon, carbon fiber, ceramic, silicone, polymers such as for examples, PTFE, nylon, Dacron, polyacrylate esters like polyester, polyurethane, polybutylene, polypropylene, polyetheretherketone, polyaryletherketone, or combinations thereof.

In one preferred embodiment, the transcutaneous component comprises carbon fiber reinforced thermoplastic resin called polyetheretherketone (carbon/PEEK).

The transcutaneous component may contact the external environment at one end, such as, for example, a prosthetic device, medical device, etc. and at the other end, the transcutaneous component may contact bone, muscle, blood vessels, nerve, organ or other tissue or combinations thereof. In various embodiments, the transcutaneous component may contain an antimicrobial agent, growth factors (such as to grow new blood vessels), or growth inhibitors or combinations thereof.

The transcutaneous component may include a sleeve running with the transcutaneous component and surrounding all or a portion of the component. In one embodiment, the sleeve runs normal to the plane of the skin/dermal anchor/epidermal component. Typically, the sleeve contacts the porous layer and/or the substantially non-porous synthetic layer. In various embodiments, the sleeve may be coated on to the transcutaneous component or attached by biocompatible cement or glue. The sleeve provides additional mechanical support to the transcutaneous component, porous layer and/or the substantially non-porous synthetic layer. In various embodiments, the sleeve comprises silicone, polyacrylate esters like polyester, polyurethane, polybutylene, polypropylene, or combinations thereof. In various embodiments, the sleeve is biocompatible, non-degradable and/or permanent. In various embodiments, the sleeve may also be integrated into the distal pylon providing an in-growth path for patient's skin. In various embodiments, the sleeve and/or pylon is of a sufficient porosity to allow the neodermis and dermis to grow into it. In various embodiments, the sleeve and/or pylon act as an attachment point for the skin. In various embodiments, the pylon has a groove cut in it and incorporates the sleeve in it. In various embodiments, the sleeve comprises a mechanical feature, such as for example a lock, which may be concave or convex that holds the sleeve and/or pylon in place.

In a preferred embodiment, the transcutaneous component comprises a pylon that is capable of receiving a prosthetic device at one end and bone at the other end. The transcutaneous component may be in two separate components, such as for example, a distal component and a proximal component. The proximal component and the distal component connect to each other, for example, by trunion or threaded joint. The proximal pylon may connect to bone by biocompatible cement, adhesive, rods, or other means that allows osseointegration with reduced tendency to generate the immune or inflammatory response and provides more natural loading of the host bone. The distal pylon component contacts the environment and is capable of receiving a prosthetic device. In various embodiments, the distal pylon contains a load-bearing region that is impact resistant, and/or damage tolerant. In various embodiments, a portion of the distal and/or proximal pylon surface has pore sizes of at least about 50 to about 350 microns for osteointegration and/or skin tissue integration into the pylon.

In one embodiment, the transcutaneous component contacts a sensor placed in the dermis or beyond the dermis, an electrical lead contacts the sensor and runs through the transcutaneous component where it can be connected to a power supply. In this embodiment, the sensor can monitor a physiological parameter including, but not limited to, electrical impulse, oxygen saturation, or glucose level.

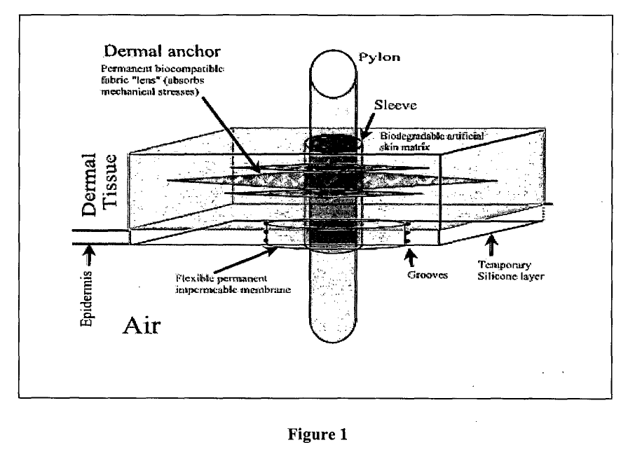

FIG. 1 illustrates one preferred embodiment of the wound closure system. This figure illustrates the collagen-GAG biodegradable porous matrix that regenerates the dermis, the non-porous silicone temporary layer that temporarily replaces the epidermis, the permanent impermeable membrane contiguous with the silicone temporary layer, the permanent porous non-degradable biocompatible dermal anchoring material that is embedded in the collagen-GAG layer, permanent non-degradable sleeve surrounding the transcutaneous component such as a pylon, catheter, etc, and the dermal anchor and the permanent membrane.

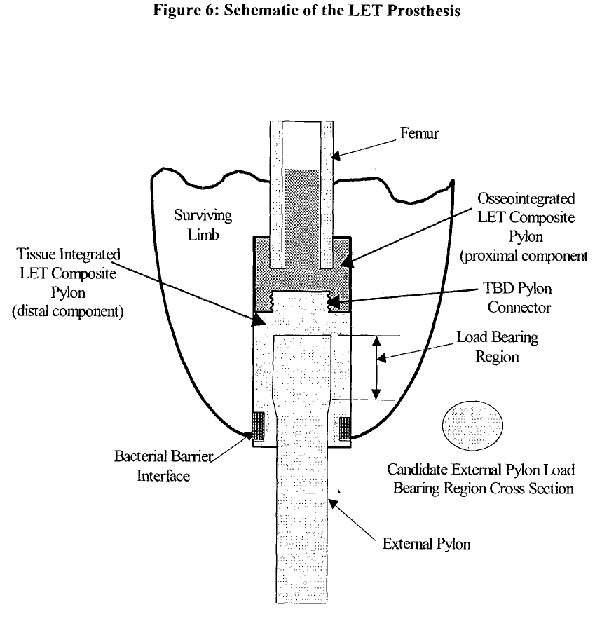

FIG. 6 illustrates one preferred embodiment of the wound closure system. A skin interface is provided containing the substantially non-porous synthetic layer contacting the porous layer and a pylon which is the transcutaneous component having a distal and proximal component. The distal component comprises a load-bearing region that is capable of receiving an external pylon such as the kind that is part of a prosthetic device. The pylon also comprises a proximal component that is capable of being attached to bone and is designed for osseointegration. These components may optionally contain antibiotics, growth factors as well as growth inhibitors.

### Wound Closure Kits and Methods

In various embodiments, a wound closure kit is provided, comprising: a porous layer comprising a collagen material; and a substantially non-porous synthetic layer contacting the porous layer, the porous layer and substantially non-porous layer are capable of receiving a pylon and providing wound closure by allowing growth of neodermal tissue. In various embodiments, the kit includes one or more containers, as well as additional reagent(s) and/or ingredient(s) for performing any methods of the invention. The kit may also include instructions for using the wound closure system. The kits may include the transcutaneous component or may be provided without the transcutaneous component.

In various embodiments, a method for providing wound closure surrounding a transcutaneous component is provided, comprising: applying a wound closure system that allows growth of neodermal tissue to a wound, the wound closure system comprising: a porous layer comprising a collagen material; a substantially non-porous synthetic layer contacting the porous layer; and a transcutaneous component surrounded by the porous layer and the substantially non-porous synthetic layer. For example, in one preferred embodiment, when the transcutaneous component is a prosthetic device, the prosthetic device is attached to the bone by means known in the art, such as for example, by cement. The surgical wound is closed using the wound closure system containing the porous layer- and the substantially non-porous synthetic layer. After about 7 to 10 days, the neodermis begins to re-grow in the porous layer and surrounds the dermal anchor. After the neodermis is fully developed, which usually takes about 2 to 3 weeks, the non-porous layer, such as for example, the temporary silicone layer is removed from the porous layer and an autologous skin graft is applied to the wound. Alternatively, the epidermis may be allowed to grow from the wound edges. The autograft may include keratinized or non-keratinized epidermis, or mucosal epithelium. The graft may consist of minimally manipulated or tissue cultured autologous cells. After the graft is applied, the wound closes and is fully healed after about one week. The transcutaneous component, for example, a prosthetic device, now has functioning epidermal and dermal tissue around it and the prosthetic device is attached to the bone.

### EXAMPLES

The examples below describe a wound closure system that will provide a permanent biological barrier at the skin implant device interface.

### Example 1

The following table summarizes the experimental plan

| Experimental | | | |
|---|---|---|---|
| Sequence | Primary objectives | Other Objectives | Models |
| Phase I | Feasibility and | Secondary objective of | Well characterized (for dermis) |
| | biomaterials selection | preliminary observation | animal model that does not need |
| | for dermal component. | of epidermal junction as a | for further development, but is |
| | | guide to our Phase II | limited to acute phase of wound |
| | | program. | healing. |
| Phase II | (1) Further | At the conclusion of | Swine model and further |
| | optimization of | Phase II, we expect to | development of this model, which |
| | materials and design of | have experimental data | will enable the observation of |
| | dermal component and | on prototypes of a | acute and chronic responses over |
| | (2) Materials selection, | complete transcutaneous | several months as well as the |
| | design and | system to support a | evaluation of prototypes |
| | optimization of the | product development | containing both dermal and |
| | epidermal component. | program. | epidermal components and that are |
| | | | completely transcutaneous. |

### Specific Aims

The overall, long term, objective of our collaborative research program is to develop a clinically useful Lower Extremity Transcutaneous (LET) prosthesis. The LET is a structural system that can be attached to an amputee's surviving natural bone to provide a direct, load-bearing path through the skin to an external prosthesis. The enabling technology for the LET, a high performance transcutaneous port (HPTP) that can provide a long-lasting skin/pylon interface that will reduce the rate of superficial and deep infection to clinically acceptable levels, is the objective of this experimental sequence (Phases I and II).

Of course, an improved transcutaneous port will have many clinically important and economically valuable applications. We focus on the prosthesis objective because it would mitigate a severe and frequent morbidity and contribute substantially to the quality of life of amputees. This focus also allows us to direct our initial product development projects on optimizing the performance of our device, without the severe competitive constraints due to manufacturing costs that would apply to an improved peritoneal port or indwelling catheter, for example.

Previous approaches to this classical medical technology problem have been directed at optimizing properties of biomaterials. However, the skin is an organ system, with dermal and epidermal tissue components. To overcome the limitations of existing transcutaneous ports, we propose a systems approach to engineering the HPTP, with functional requirements for both dermal and epidermal components. Our approach parallels the successful tissue-engineering design principles of Burke and Yannas¹, which addressed the physiology of both the dermis and epidermis in the design of their clinically successful artificial skin^{2,3,4}. Furthermore, we apply the clinically proven artificial skin technology to our new clinical application.

This Phase I experiment is focused more narrowly on biomaterials selection to achieve a key functional requirement of our design: to establish stable integration of non-degradable, structural, biomaterial fibers with the neodermal tissue that is induced by the biodegradable artificial skin after implantation of the device.

The Specific Aims of the Proposed Phase I Research are to:

Confirm, in a well-characterized small animal model, our hypothesis that suitable non-degradable biomaterial fibers will not interfere with formation of neodermal tissue by artificial skin when they are embedded in the degradable matrix component of the artificial skin. The control material for this confirmation will be unmodified artificial skin.

Confirm in the small animal model our hypothesis that fibers embedded in artificial skin will have a decreased acute foreign body response when embedded in neodermal tissue in comparison to embedding in an open wound. Identify experimentally the critical design parameters for biomaterial bulk and surface chemistry, added biomolecules, surface texture, fiber diameter and fabric weave that optimize the dermal integration performance of the fibers, as demonstrated by good neodermis formation and insignificant foreign body response to the fibers.

Design prototypes that demonstrate a viable structural approach and fabrication path to the dermal anchor, epidermal junction, and pylon system. Based on these prototype designs, fabricate test articles with dermal anchor and epidermal junction components (without pylon) for preliminary testing in small animal model.

Demonstrate the ability of the small animal model to characterize the in vivo epidermal interaction with these integrated devices as well as the longer-term biocompatibility of the dermal component over a period of up to 6 weeks. We will also characterize the performance of this integrated device, including the development of any chronic inflammatory responses.

Our key criterion for Phase I success is a biomaterial construct that stably integrates with dermis during the acute healing phase. Thus, Specific Aims 1, 2, and 3 represent the critical feasibility test of our concept and the primary objective of Phase I. Specific Aims 4 and 5 address collection of preliminary data for the epidermal component of our design, a secondary goal.

### Medical Significance of the LET

Loss of a limb or part of a limb is a dramatic event that changes lives. It occurs for many reasons, most commonly as a result of trauma or as part of the treatment of malignancy or infection. In 1996 there were 1,285,000 amputees in the US. The incidence in 1996 was ˜4.9 per 1,000, or roughly 1 in 200 persons (National Health Interview Survey, Office on Disability and Health, National Center for Environmental Health, Centers for Disease Control and Prevention, Atlanta Ga.). In addition, children born with congenital limb deficiencies represent an additional group of persons similarly affected. An estimated 1 in 2,000 babies are born with all or part of a limb missing, ranging from a missing part of a finger to the absence of both arms and both legs⁵.

Prosthetic replacement is the most common option for most limb losses. For any prosthesis to function, it must interface with the residual limb to adequately transfer the loads of physical support, motion and control. This is traditionally achieved through an intimately fit socket. The socket is shaped to contain the volume of the residual limb segment while distributing interface stresses in a manner tolerated by the tissues. Practically, this balance of loads in the dynamic situation of the prosthesis is extremely difficult to optimize, and the result is often socket induced pain and reduced function, even in cases considered to be quite successful from the standpoint of conventional prosthetics. While many amputees function with the traditional socket style prosthesis, this system has many inadequacies. Pressure points result in skin breakdown and discomfort. Socket pain results in the inability to wear the prosthetic device over long periods of time. Skin reaction and breakdown is a frequent problem with amputees requiring time out of the prosthetic device, treatment for infections, ulcerations, or even surgery. Many patients simply cannot tolerate socket style prosthetic devices⁸.

An important clinical advance offered by the Lower Extremity Transcutaneous (LET) prosthesis is the elimination of the prosthetic socket, hence the elimination of the majority of the common problems of the prosthesis user. For centuries, mankind has envisioned directly attaching artificial limbs transcutaneously through the skin directly into the skeleton. Direct skeletal loading has the obvious difficulty of designing a suitable implant that is biocompatible, distributes load effectively over the skeletal section to which it is attached, and allows for passage through the skin so that an artificial limb can be attached.

Where transcutaneous direct skeletal attachment of artificial devices has succeeded, it has allowed individuals with limb loss to wear a prosthetic device without the detrimental affects of the socket on the amputation stump. The most successful clinical attempt is the osseointegration titanium prosthetic implant developed by Professor P. I. Branemark of Gothenburg, Sweden⁶. In early testing, patients have marveled at the tremendous advantages of direct skeletal attachment. Personal interviews with one patient compared his old socket style prosthesis to a horse and buggy and the new direct skeletal attachment prosthesis to a spaceship⁷. The patient notes that the old prosthesis felt like it was something that he put on, whereas the new prosthesis was part of him. He commented on the improved suspension, the ease of control of the device, the instant feedback of where the device was in space, and the proprioceptive knowledge of when the device was in contact with an object in the external environment. This antidotal response has also been mirrored in controlled clinical studies where the bone-anchored prosthesis had significantly better perception than the socket prostheses.⁸

While early studies have substantiated the dramatic impact direct transcutaneous skeletal attachment devices can afford amputees, the current system has many problems. Unfortunately, there is no true biologic integration of the skin to the prosthesis. Without a biologic interface, bacteria can migrate down the surface between the skin and the device and inevitably, infections occur in time frames from several weeks to several years. Thus, superficial infection is considered a permanent aspect of the titanium implant, and deep infections have also been reported.

### HPTP Background

The enabling technology for a LET is a more permanent transcutaneous access. Our technical approach to the high performance transcutaneous port (HPTP) is based on modification of the wound physiology that is the immediate response to the implantation of a biomaterial. Our primary tool is the clinically successful artificial skin graft technology, described below. This artificial skin technology is supplemented by recent characterizations of biomolecules that modify the wound environment.

Permanent transcutaneous access will have many valuable medical applications in addition to the LET. However, developing the transcutaneous technology for the LET application has advantages: (1) it provides enabling technology for an unsolved serious medical need, and (2) the design can be driven primarily by performance criteria instead of manufacturing cost, as would be the case for vascular or peritoneal access.

### Biomaterial Implants

The compatibility of biomaterials with blood and tissue is critical for the successful function and longevity of medical devices. Hip joints loosen and need replacement at rates that have not changed in the past 50 or more years, despite extensive investment in design. Intraocular lenses need replacement at rates of 20-30%. Vascular grafts fail to endothelialize in patients despite several successful approaches in animals. Cardiovascular stents suffer from restenosis. More relevant to the current proposal, the use of in-dwelling catheters results in several hundred thousand systemic infections and as many as 50,000 deaths yearly in the US.

The chief problem with implanted biomaterials is the “foreign body reaction” (FBR) in which the tissue rejects the presence of the material. Beginning with an initial non-specific absorption of serum proteins to the biomaterial surface, the otherwise normal role of inflammation in the healing tissue becomes prolonged and leads to a chronic inflammatory state.

A characteristic of implant healing is the fusion, at the material surface of macrophages to form foreign body giant cells (FBGC). These mulinucleate cells remain at the surface indefinitely and are capable of maintaining the state of chronic inflammation of the tissue. They also are capable of degrading the material surface causing additional problems. The end result of implant healing is that the resulting tissue forms into a fibrous avascular capsule surrounding implanted biomaterials.

### Fundamental Skin and Skin Wound Physiology

The skin is a complex organ composed of two main layers: dermis and epidermis. An intact epidermis provides a barrier to microbial invasion or loss of fluid and other functions of normal physiological homeostasis. The dermis provides the essential mechanical functions of skin due to the strength and elasticity of its collagen- and elastin-rich extracellular matrix. The dermis is also a vascularized tissue that provides nutrition to both the dermis and epidermis, and its transport of immune system components is an essential part of the barrier function of the skin. The physiological response to a clean full thickness skin wound is to initiate a tissue repair process.^{9,11} (“wound healing by second intention”¹²). This tissue repair response is characterized initially by inflammation, edema, and fluid loss. Following the initial inflammatory stage, mesenchymal cells proliferate to form a richly vascularized “granulation tissue” in the wound bed, and contraction of the wound brings the wound margins to close apposition. Migration of epidermis from the wound edges closes the wound and the vital barrier functions of skin are restored. However, there are undesirable, long-term, consequences of this natural wound healing process since it does not result in the formation of normal dermis. Wound contracture and the formation of permanent, inflexible, scar tissue can result in partial or complete immobilization of joints, chronic fragility of the overlaying epidermal tissue, discomfort, and unacceptable cosmetic appearance.

### Skin Replacement Surgery

Because there are several skin tissue engineering technologies^{9,16}, and because their clinical utilities are frequently misunderstood, the following information is presented in some detail. Skin lesions that are not expected to heal spontaneously with good clinical outcome are treated by skin replacement surgery. Skin replacement surgery is a two-step procedure: The first step of skin replacement surgery is surgical excision of the lesion and any necrotic tissue or microbial contamination, resulting in a clean surgical skin wound. The second step in skin replacement surgery is the application of skin autograft to the clean surgical skin wound. The physiological response to skin autograft applied to a clean surgical skin wound comprises two phases: (1) an immediate wound closure response followed by (2) dermal tissue engraftment and epidermal tissue engraftment.

The immediate wound closure physiological response differs critically from the open wound physiological response. It is characterized by immediate restoration of some of the physiological functions of skin including an immediate reduction in wound inflammation, pain, and fluid loss. Granulation tissue is not formed, and wound contraction does not occur.

The end result of a skin autograft is a “healing by first intention,” in which healing occurs directly, without intervention of granulations, and the lost skin is permanently replaced by intact healthy skin with normal tissue architectures of both dermis and epidermis (without significant scar or contracture).

### Skin Tissue Engineering

For skin wounds where there is not sufficient donor tissue to perform a skin autograft, a substitute for skin autograft is needed to accomplish skin replacement surgery. The performance requirements for a substitute for skin autograft are that, when applied to a clean surgical skin wound, it produces a wound healing by first intention, including an immediate wound closure physiological response and the permanent replacement of the lost skin with intact healthy skin with normal tissue architectures of both dermis and epidermis.

A successful approach to this problem is the tissue-engineering principles and technology that were successfully used by Burke and Yannas¹ to design the clinically successful^{2,3} and commercially available “Integra Artificial Skin.” Burke and Yannas divided the clinical requirements for an artificial skin graft into two stages:^{1,13} (1) immediate physiological closure, of the wound (which is characterized by a lack of inflammation, pain, wound contraction, or formation of granulation tissue), and (2) a permanent vascularization of the graft and regeneration of the dermal and epidermal skin layers, without introducing fibrosis, scar or contracture.

Artificial skin comprises a porous collagen-glycosaminoglycan (GAG) dermal regeneration layer and a silicone temporary epidermal-substitute layer that is firmly bound to it. The silicone layer of the artificial skin substitutes for the epidermis to provide a barrier to microbes and moisture loss until vascularization of the dermal layer is complete. The collagen-GAG dermal layer provides a scaffold for ingrowth of fibroblasts and vasculature without inflammation or formation of granulations, after which final definitive closure is achieved by removing the silicone layer and covering with an autograft of epidermis. The clinical utility of an artificial skin graft for treating surgically excised wounds has been demonstrated in clinical trials on burn patients.^{2,3,4} Artificial skin has since been approved by regulatory authorities in the United States and the European Union and is available as a commercial product (Trade name: INTEGRA® Dermal Regeneration Template; manufactured by Integra Lifesciences Corporation and distributed by Ethicon, Inc. Division of J&J.)

The bilayer artificial skin graft functions by providing an immediate physiological closure of the wound that inhibits inflammation and minimizes the formation of granulation tissue¹⁴, wound contraction, fluid loss, and the systemic effects of an open wound². The porous collagen-GAG layer achieves the stage 1 functional requirements for biocompatibility and low inflammation, and contractile cells are not observed in either stage of healing¹⁴. The highly hydrophilic biomaterial and the porous design make the artificial skin adherent to the wound bed¹. The silicone layer contributes the stage 1 requirements for low moisture transmission and impermeability to microorganisms, as well as the appropriate tear strength and handling characteristics. Because the silicone layer is not degradable under physiological conditions, these properties persist until a surgeon removes the layer in a stage 2 procedure. The stage 1 initial wound closure is followed by vascularization of the dermal layer and regeneration of a permanent, autologous, dermal tissue; the original material from the dermal re-generation layer is degraded and remodeled over a period of 1 or 2 months.

Upon adequate vascularization of the dermal layer (typically about 2-3 weeks), a stage 2 surgical procedure is performed in which the temporary silicone layer is removed, and a meshed layer of ultra thin epidermal autograft is placed over the neodermis. Cells from this epidermal autograft migrate and grow to form an intact, normal, epidermis, thereby completing the stage 2 closure of the wound and regenerating a functional, autologous dermis (“neodermis”) and epidermis. Analysis of biopsy samples from patients in a clinical trial showed no evidence of scar formation,¹⁴ and an analysis of regenerated skin in reconstructive surgery patients showed that newly synthesized collagen was histologically indistinguishable from normal dermal collagen.¹⁵

These observations demonstrate that the Burke/Yannas artificial skin can achieve a wound healing by first intention, demonstrated by a wound closure immediate physiological response and the permanent replacement of the lost skin with intact healthy skin, with normal tissue architectures of both dermis and epidermis¹⁴ (except that epidermal appendages such as hair are not regenerated).

Healing of Open Wounds vs. Implantation of Biomaterials

The parallel between the healing of open wounds and the implantation of biomaterials (both are driven by inflammatory processes and result in scar formation) is the basis of our hypothesis: that the ability of artificial skin technology to heal full thickness skin wounds “by first intention”¹² (characterized by lack of inflammation, granulation tissue and scar tissue) can bring about a functional integration of a suitable biomaterial with dermal and epidermal tissue.

### Other Skin Substitute Technologies

Table 1 is based on an ASTM Standard F2311-03,¹⁶. It is intended to illustrate that of various skin tissue engineering technologies, the Burke and Yannas artificial skin and a cultured bilayer skin substitute,¹⁷ both of which are based on a collagen-GAG substrate, can substitute for skin autograft for full thickness wound closure. Cultured allogeneic human fibroblasts provide only a temporary wound closure, similar to allograft. Cultured autologous keratinocytes provide a permanent wound closure, but do not replace dermis, which is critical to normal skin function.

TABLE 1
Classification of Skin Substitutes

| Classification | Technology | Comments |
|---|---|---|
| Substitute for skin | Human foreskin | Appligraft ® and Dermagraft ® These |
| allograft for skin | fibroblasts cultured in | materials can be used to accelerate the |
| allograft therapy | vitro in or on a matrix | healing of non-healing wounds. |
| | when used to treat a skin | |
| | ulcer.¹⁸ | |
| Substitute for skin | Human foreskin | “TransCyte ®” is a temporary wound |
| allograft for skin | fibroblasts cultured on an | closure for full or partial thickness |
| replacement | occlusive membrane.¹⁹ | wound; for a full thickness wound, it |
| therapy | | must be replaced by autograft. |
| Substitute for skin | Bilayer of porous | “Integra ® Artificial Skin” |
| autograft for skin | collagen-GAG substrate | |
| replacement | and silicone membrane | |
| therapy | when used in conjunction | |
| | with an epidermal | |
| | autograft². | |
| | Cultured bilayer of | Construction of a transcutaneous |
| | autologous fibroblasts | device using in vitro culture of |
| | and keratinocytes on | autologous cells may be technically |
| | porous collagen-GAG | possible, but is more complex to |
| | substrate.¹⁷ | commercialize than our approach. |
| Substitute for | Sheet of cultured | “Cultured Epidermal Autograft |
| epidermal autograft | autologous | (CEA)” Application of cultured |
| for permanent | keratinocytes.²⁰ | epidermal cells to a full-thickness skin |
| wound closure | | wound does not regenerate a dermal |
| | | layer; the clinical result is that the new |
| | | epidermis, while autologous and |
| | | permanent, may overlay scar tissue and |
| | | be fragile. |
| Substitute for | Decellularized human | Decullarized dermis does not close |
| dermal autograft | dermis. “Alloderm ®” | wounds. |
| for reconstructive | | |
| surgery procedures | | |

### Control of the Foreign Body Response

### Fiber Parameters

Biocompatibility of porous and fibro-porous biomaterials is influenced by the microarchitecture of the implant,²¹ with fine monofilament materials performing better than thicker fiber materials. For example, Bernatchez,²² showed reduced cell spreading in an in vitro macrophage cell culture model for 12 μm gold fibers compared with 25 μm fibers, and for thin-fibered, nonwoven polybutylene/polypropylene (2 to 12 μm in diameter fibers) materials and nonwoven polyester (10 to 12 μm in diameter fibers) materials compared with thick-fibered woven polyester (40 μm in diameter fibers) materials and woven nylon (38 μm in diameter fibers) materials. For fabrics, internodal distances and open area percentage are also important. Clark demonstrated a strong correlation between inflammatory tissue reaction and the ratio of the percentage of open area to fiber diameter.²³ Jansen,²⁴ using sintered metal fiber-web materials, found that fibrous capsule thickness was reduced for higher porosity implants.

The University of Washington Engineered Biomaterials consortium has also developed unique fibroporous meshes that do not lead to a FBR upon implantation.²⁵ The fibers, produced by electrospinning, are of very small diameters (<10 micron) and do not allow macrophages and FBGC to adopt a chronic inflammatory state. The elasticity of the fibers is also important for biomechanical influences on the healing tissue.

### Composite Materials

One of the engineering challenges of the dermal and epidermal components of the transcutaneous access device is to prevent abrupt mismatches between the compliance of skin and compliance of the device. A similar problem, to be addressed in Phase II, is to prevent abrupt mismatches between compliance of the pylon and that of bone; it is our hypothesis that such mismatches contribute to the limited lifetime of hip and joint implants. We believe that our expertise in advanced composites will help us solve such problems.

Advanced composites are highly versatile and can be engineered for specific structural and morphological applications. Composites are multiphase (usually two) materials comprising a stiff, strong, oriented reinforcing phase embedded in a relatively soft, weak matrix phase. Well-established compositing and laminating techniques permit the designer to tailor the inherently anisotropic response of composites to achieve the optimal structure that satisfies the directionally sensitive stiffness and strength requirements of a particular application. The mechanical response of the composite is determined primarily by the type of fiber, fiber length (discontinuous or continuous), fiber architecture (direction and volume fraction), and fiber/matrix microstructure. Not only can the structural response of composites be tailored but also the material characteristics such as porosity, morphology (through fiber selection, architecture, and processing), and interstitial characteristics can also be designed (within limits).

This ability to locally tailor the properties of a composite provides the opportunity to design an integral interface for the artificial skin scaffolding system where the geometry as well as the compliance of the composite can be varied such that the material can closely mimic the mechanical properties of skin.

Because composite materials can be made from a large variety of fibers and matrices, the requirement of biocompatibility can be accommodated by selecting constituent fibers and matrix materials which are biocompatible. We propose to use a carbon fiber reinforced thermoplastic resin, polyetheretherketone ²⁶ (carbon/PEEK) as the LET Prosthesis composite structural material. Carbon/PEEK is lightweight (˜37% of titanium) and has demonstrated excellent biocompatibility^{27,28} and is currently being used in long term human implants that have received FDA marketing approval²⁹.

### Phase II Research Plan

The Phase I experiments necessarily address the transcutaneous device only in a preliminary way. In Phase II, in addition to further optimization of the materials and design of dermal component, we will perform the materials selection, design and optimization of the epidermal component. Specific aims 4 and 5 of Phase I are pilot studies to evaluate the junction of epidermis advancing from the wound edge with a membrane (silicone initially) that is mechanically combined with the dermal component. This experiment will model the critical external side of a transcutaneous device and may allow some of the Phase II research to be conducted with the small animal model. (We believe that the internal junction of the skin interface with the pylon and of the pylon with subcutaneous tissue represent less of a technical challenge.) Phase II research will utilize a swine model and we expect to further develop this model, which has previously been used only for artificial skin studies. The swine model will enable the observation of acute and chronic responses over several months as well as the evaluation of realistic prototypes containing both dermal and epidermal components.

In Phase II experiments, we plan to simulate the pylon so that the prototype device is completely transcutaneous. We don't believe the topology of this prototype will differ significantly from one that includes a pylon. At the conclusion of Phase II, we expect to have experimental data on prototypes of our complete dermal/epidermal system will provide a basis for a phase III program to complete the product design, manufacturing methods, and collection of preclinical and clinical safety and efficacy data that regulatory submissions, and commercialization of the LET.

### Research Design and Methods

### Design Plan for the HPTP

We propose a systems approach to engineering the HPTP, with functional requirements for both dermal and epidermal components. Our approach parallels the successful tissue-engineering design principles of Burke and Yannas, which addressed the physiology of both the dermis and epidermis as well as the physiology of wound healing in the design of their clinically successful artificial skin graft. We expect this approach to enable the development of a long term, efficient infection/bacterial barrier at the skin interface that will reduce the rate of superficial and deep infection to clinically acceptable levels.

### Functional Requirements of the HPTP

Burke and Yannas divided the design requirements of the artificial skin graft into two stages of wound closure¹: (Stage 1) a requirement for immediate physiological closure of the wound and (Stage 2) a requirement for permanent vascularization of the graft and regeneration of the dermal and epidermal skin layers, without introducing fibrosis, scar, or contracture. For a permanent transcutaneous port, the adapted design requirements are:

1. After implantation of the device, an immediate physiological closure must be achieved (i.e., without significant inflammation); and

2. A permanently healed wound, with intact dermal and epidermal tissue, without a continuing inflammation or formation of significant scar tissue. Additional design requirements for the HPTP address the requirements for permanent non-degradable components that penetrate the epidermal and dermal layers of skin, and the continuity of the physiological and mechanical interfaces between the device and intact skin:

3. Epidermis should be supported by healthy dermis that retains its functions of nutrition, pericrine signaling, basement membrane and access to circulating cellular and humoral immune components. This healthy dermis must be maintained under the point of junction between epidermis and biomaterial.

4. The device/dermis junction should be able to absorb the mechanical stresses between normal skin and device.

5. The junction between epidermis and the device should not be subject to significant mechanical stress and the migration of epidermis under the biomaterial components must be prevented.

6. In case of failure, the device should be replaceable without removal of the pylon.

### Design for the HPTP

Our hypothesis is that the natural physiological response is not conducive to the integration of a biomaterial and that rapid physiological wound closure and the controlled synthesis of permanent new connective tissue can enhance the biocompatibility of a properly designed porous biomaterial and also generate mechanical continuity of vascularized connective tissue with it.

FIG. 1 illustrates the design concept, which adapts the proven biomaterials developed by Burke and Yannas for the artificial skin graft to our new requirements. There are five biomaterial components: Collagen-GAG biodegradable porous matrix; Silicone temporary layer; Permanent non-degradable impermeable biocompatible membrane contiguous with the silicone temporary layer; Permanent porous non-degradable biocompatible dermal anchor that is embedded in the collagen-GAG layer; Permanent non-degradable sleeve which provides mechanical interfaces with pylon or catheter, etc.), the dermal anchor and the permanent membrane; and optional antimicrobial agent release system (such as the commercially available Biopatch®), if needed (not shown).

The biomaterial disk will be designed to have graded mechanical properties that match the compliance of the dermis at its outer circumference to minimize dermal stress but stiff near the center to minimize strain under the epidermal junction. The membrane may be metal or polymer and will have an appropriate composition on the underside to integrate with dermis and inhibit epidermal migration.

The engineering design of the epidermal component will be based on different considerations than the dermal component. Bulk porosity at the air interface is undesirable and would create paths for microbial access, but as discussed below, the dermal contact side must also have appropriate surface chemistry and texture for dermal integration. We tentatively expect this component to take the form of an impermeable membrane (metallic or polymer) fused to the dermal anchor layer. In that way the fibrous structure of the dermal material will be located where it can help inhibit epidermal migration.

### Intended Function of the HPTP

The HPTP will be implanted in a surgically prepared excised wound. The collagen-GAG layer will fill the wound and the silicone layer will be sutured or taped to the intact skin. The device will provide a stage 1 physiological closure. As with artificial skin grafts, new dermal tissue will be synthesized in the collagen-GAG matrix. Our hypothesis is that the wound healing physiology created by the artificial skin components will be conducive to the stable integration of the dermal anchor. We expect that there will be a minimal and transient giant cell response to the dermal anchor and that newly deposited extracellular matrix will encapsulate the fibers of the dermal anchor and mechanically connect it with the neodermis. After formation of a neodermis, the silicone layer will be removed, and (if necessary) the neodermis will be seeded with epidermal tissue. Alternatively, the epidermis may be allowed to grow from the wound edges. The autograft may include keratinized or non-keratinized epidermis, or mucosal epithelium. The graft may comprise minimally manipulated or tissue cultured autologous cells. We expect that a properly designed dermal anchor will not interfere with the normal epidermal/dermal physiology and that new epidermal tissue will become confluent. Our functional requirement that healthy dermis is maintained under the epidermis/biomaterial junction is intended to ensure that a healthy epidermis will be maintained at the junction with the membrane.

One uncertainty is how securely epidermis will adhere to the membrane and provide a barrier to microorganisms, since there are no natural junctions of squamous epidermis with a transcutaneous tissue to provide a physiological mechanism for a tight junction between epidermis and biomaterial. The essence of our design is the presence of healthy dermis which will provide the critical immunological functions to protect this junction.

### Experimental Plan for Design Verification

### Biomaterials Selection for Dermal Integration

The initial design for the dermal anchor is a porous, fibrous, disk of a non-degradable biomaterial that will become encased in neodermis induced by the collagen-GAG artificial skin component. Specific Aims 1, 2 and 3 address the key functional requirements for integration of the dermal anchor during the acute healing phase (2 to 3 weeks): These aims will be studied by a combinational approach to screen for significant biomaterial parameters as well as to optimize those parameters for performance in a guinea pig wound healing model. The objectives are to achieve (1) no significant degradation in artificial performance during neodermis formation, and, (2) no significant increase in the density of macrophages and FBGC due to the inclusion of a non-degradable biomaterial.

### Design of Experiments

Our optimization methodology (Specific Aim 3) uses a sequence of designed in vivo screening and optimization experiments.³² This response surface modeling methodology uses the fewest number of experiments to select from a large set of parameters (chemistry, size, spacing, etc.). Fractionated factorial designs are used to identify the most significant parameters and response surface experiments are used to optimize the most critical parameters. In each experiment we will construct test articles that are modifications of artificial skin in which biomaterial fibers are embedded in the collagen-GAG matrix. The test articles will be implanted in using our well established guinea pig dermal wound model, and, after necropsy, we will examine the tissue responses to the test article by histology. Critical parameters that will need to be chosen include bulk and surface chemistry (possibly including specific cellular adhesion molecules), surface texture and/or open cell porosity (both void volume and mean pore size), and geometry. (Appropriate porosity strongly influences the fibrous encapsulation of implanted biomaterials.³³)

Interference of the biomaterial with neodermis formation will be measured by the scoring system that is described in Methods. Paired comparison of the test articles with control artificial skin on the same animal contributes to the statistical power of this scoring system. (FBGC are occasionally seen in unmodified artificial skin.) We hope that after screening experiments identify materials that do not interfere with neodermis formation, the paired comparisons with control artificial skin can be eliminated. Paired comparisons of the neodermis quality and foreign body reaction can then be made directly between prototypes, to increase the statistical power of the optimization experiments. Alternatively, the control wound site can be used to compare the behavior of biomaterials embedded in artificial skin with the same fibers in open comparison wounds in order to confirm our hypothesis that the artificial skin matrix can reduce the foreign body reaction with a biomaterial.

An example of a likely sequence of 4 animal experiments are shown in Table 2:

TABLE 2
Possible sequence of experiments

| Sequence | Experimental Design | Design Outputs |
|---|---|---|
| 1 | One dimensional experiment, 6 test | Choose best fiber candidates based on |
| | articles of fibers in artificial skin, n = | minimum effect on neodermis |
| | 5 animals per group, paired | |
| | comparison with artificial skin, 3 | |
| | week time point | |
| 2 | Fractional factorial, 2 levels of | Identify most important fabric variables |
| | three parameters, e.g., diameter, | and confirm hypothesis that appropriate |
| | surface, spacing, 4 test articles of | conditions have minimal effect on |
| | fabrics in artificial skin, with n = 6, | neodermis. |
| | at 3 week time point | |
| 3 | Full factorial on 2 parameters plus | Optimize dermal parameters and test |
| | center point = 5 test articles of | epidermal model, verify utility of |
| | fabrics having epidermal | model for dermal behavior at 4 weeks, |
| | component attached, plus open | characterize epidermal interaction with |
| | wound control group at center | epidermal component and verify utility |
| | point, n = 5 at 4 week time point | of model for study of epidermal |
| | | interactions |
| 4 | Fractional factorial on 3 | Validate dermal design and identify |
| | parameters of epidermal | most important variables for epidermal |
| | component material and | component based on best contact and |
| | attachment to dermal component | minimal undermining |

### Assays and Biomaterial Selection Criteria

Our objective for successful incorporation of non-degradable fibers is to achieve minimal degradation in the quality of newly synthesized dermal tissue (in comparison with artificial skin without added biomaterials), minimal Foreign Body Giant Cells (FBGC) associated with the fibers, and thin and stable encapsulation of the biomaterials fibers with connective tissue extracellular matrix similar to and integrated with those of the bulk of the newly synthesized dermal tissue.

We have the advantage of a well characterized guinea pig model to evaluate the critical first three weeks of neodermis formation. During this acute healing phase, artificial skin becomes fully infiltrated with vascular connective tissue and degradation of the collagen-GAG component will be underway. Inflammatory responses to the biomaterial fibers can be recognized by giant cell responses and alterations in cellular architecture.

Thus, the criteria for selection of biomaterials for stable dermal integration are (1) that they do not interfere with the wound closure physiology and neodermis formation by the collagen-GAG component during the acute healing phase and (2) that do not induce an acute foreign body reaction on the biomaterial fibers during this acute healing phase. Acute wound healing in this model takes place over approximately three weeks, and a suitable time point to terminate the in vivo experiment would be about two weeks. The histological appearance of an artificial skin wound at this time is well characterized.

The opinion of an experienced histologist should be adequate to characterize foreign body response to the biomaterial fibers during initial screening experiments. We will then evaluate more quantitative measures that count FBGC as well as apoptotic FBGC. More precise measurements will be made during optimization experiments, including counts of FBGC and apoptotic FBGC. We will also evaluate confocal microscopy as an alternative method of counting FBGC on biomaterial fibers, as described in Methods.

### Test Articles

Most of our initial material choices for the fibers of the dermal component will be fibers of medically proven biomaterials: polyester fiber, polyamide fiber, PFTE fiber, Expanded PFTE fiber, Poly (Hexafluoropropylene-VDF), PEEK, polyurethane. Additional chemistries will be tested, if needed. Although these materials have established various degrees of biocompatibility, we believe that the artificial skin matrix will substantially modify the interaction with tissue. Initially, we will test the fiber alone, so that fiber spacing will not be a factor, and we will use commercial sutures, when available, to ensure appropriate surface properties.

The fibers will be embedded in an artificial skin matrix as described in Methods before being implanted into the dermal wounds in guinea pigs. The most suitable fibers will then be tested in fabrics. Some of these materials are available as surgical meshes, but we may need to optimize fiber diameter and spacing for our design. For example, if polyester is a satisfactory fiber, we may be able to use industrial polyester screens (Tetko, Inc.), which are available in a wide range of dimensions to pursue optimal parameters. We will need to evaluate the results at this stage in order to decide whether the expense and time to design custom fabrics will be necessary.

### Design of Prototypes

Based on the design outputs (from the above biomaterials selection experiments) for suitable biomaterial chemistry, diameters, and spacing for biocompatibility for the dermal anchor, we will develop a preliminary engineering design in collaboration with Sparta, Inc. to support epidermal integration experiments as well as to provide the design inputs for Phase II research.

The pylon, in addition to the composite design, will require some design effort in surface characteristics and attachment features of the pylon at the skin-pylon junction. The pylon design includes structural fiber selection and architecture, investigation of potential compatible skin matrix attachment features, large deformation Finite Element Analysis to assess the skin/pylon interface, and developing a fabrication approach.

After a preliminary design has been established, a detailed 3-D finite element model (FEM) of the prosthesis will be developed to analyze the detailed response of the prosthesis and the connective tissue. Loads and boundary conditions identified in Task 1 and subsequent phases including the specific requirements of the skin/pylon interface as well as the bone/pylon interface will be used to guide the design of the subcomponents and the full LET composite pylon. It is anticipated that several finite element models will be developed incorporating various geometries and/or material architectures to parametrically evaluate the design options.

The output of this would be a prototype pylon design which demonstrates a viable structural approach and fabrication path. The Phase I prototype design would be used as the baseline design in the Phase II development program.

### Biomaterials Selection and Design for Epidermal Integration.

Specific Aims 4 and 5 are to design and make model devices with both dermal anchor and epidermal junction components and characterize their interaction with epidermis. A tentative design for initial prototypes is a pad of woven or non-woven non-degradable biomaterial fibers partially embedded in a silicone membrane, with the collagen-GAG suspension embedding portion of the pad that is not embedded in silicone. These can be created by modification of the standard procedure described in Methods. For the biomaterial fibrous pad, we may try to utilize a suitable commercial mesh, if it is available with acceptable performance in our in vivo model, in order to expedite the epidermal experiments. In that case, further optimization of the fabric for the dermal layer could be then carried out in the same experiments as the epidermal component selection.

We will then test these prototypes in our guinea pig model, extending the time to 4 to 6 weeks to characterize the interaction of the epidermis with the prototype. Although the guinea pig model has previously been used to evaluate epidermal cell seeding over time periods up to 120 days,³⁴ there may be complications in interpretation of results and some model development may be required. This is because the strong contraction of guinea pig dermal wounds is not stopped by epithelialization of the wound, as it is in swine and humans.

We expect there to be time and resources to carry out only one experiment with an epidermal component. Thus, we do not necessarily expect to confirm our hypothesis that the epidermal migration will not undermine the junction with the epidermal element. Our objective is primarily to characterize the response so that Phase II experiments can be planned. Since we expect to develop and alternative swine model in Phase II, we will not be dependent on the success of this guinea pig model.

### Methods

### Test Articles Incorporating Artificial Skin Technology

The operations described below are carried out under aseptic conditions.

### Preparation Collagen-GAG Dispersion

A suspension of 0.25% w/v of fibrous collagen (e.g., from bovine hide or tendon) is dispersed by means of a suitable homogenizer in 0.05 M acetic acid at about pH 3.2 and a temperature below 20° C. Since this pH is below the isoelectric point of collagen, a viscous suspension or gel is formed as the collagen molecules swell.³⁰ Electron micrographs of fibers from this gel show that the characteristic collagen banding at 64 nm, a feature of the quaternary structure of collagen fibers, is lost. However, the collagen is not denatured, as demonstrated by infrared spectroscopic measurement of the triple helical tertiary structure of the collagen molecules.^{35,36}

A 0.1% w/v solution of chondroitin 6-sulfate is added slowly into the homogenizer to a final concentration of about 8 wt % (e.g., the volume of c6s added is 20% of the volume of collagen suspension). A precipitate is formed by ionic bonding between the cationic collagen and anionic chondroitin-6-sulfate. The precipitation reaction can be observed by a decrease in viscosity of the suspension.³⁵ The density of the mixture is increased by centrifugation. An aliquot of supernatant equal in volume to half the original volume is removed. The target density of the suspension is about 0.5% w/v. This suspension will be used to impregnate the non-degradable dermal integration fibers.

### Lyophilization and Dehydrothermal Cross-Linking

The gel is poured into trays and leveled. The trays are placed on chilled shelves of a lyophilizer. The freezing of the suspension leads to a phase separation, in which crystals of ice form one phase and compressed, hydrated collagen fibers become another. The result is a frozen, porous sponge. The cooling rate of the suspension determines pore size and shape; average pore size is one of the critical quantitative parameters affecting the biological activity of artificial skin.³⁵ Lyophilization of the frozen sponge produces a dry sponge. The pore structure of the collagen sponge would quickly collapse upon rehydration.³² An initial cross-linking of the dry sponge to preserve its porosity as well as to prevent subsequent elution of the chondroitin 6-sulfate is accomplished by extreme drying at 105° C. at below 100 mtorr for about 1-5 days. Covalent links can be formed in gelatin (and by inference in collagen) under these conditions.³⁸ The result is a sponge that does not collapse when rehydrated.³⁷ Denaturation of collagen does not take place at this high temperature because the collagen is dry.³⁹ For biomaterial components that cannot be heated to 105° C., we have alternative methods to accomplish this step.

### Coating, Rehydration, and Glutaraldehyde Cross-Linking

To form the bilayer artificial skin device, the sponge is now coated with a medical grade of silicone adhesive, and the adhesive is allowed to cure.³⁵ The sponge is then rehydrated in 0.05 M acetic acid. This acidic pH is the same as that used to form the collagen-GAG precipitate, so the ionic bonds between the collagen and the glycosaminoglycan will be maintained. Further cross-linking of the collagen component of the device is accomplished by soaking in a solution of 0.25% w/v glutaraldehyde in 0.05 M acetic acid for 24 h. The reaction of glutaraldehyde with collagen is slow at low pH. However, subsequent analysis demonstrates that cross-links are formed under these conditions. The time, concentration, and temperature parameters of glutaraldehyde cross-linking determine cross-link density, which controls the degradation rate of the collagen when exposed to collagenase, as well as the in vivo residence time of the collagen-GAG material.

### Washing, Storage and Preparation for Use

The device is washed in multiple washes of water to remove residual glutaraldehyde and acetic acid. The concentration of glutaraldehyde in the final wash should be below 2 ppm. It is not terminally sterilized. It is stored in 70% isopropanol, as a preservative. The device is prepared for use by soaking in isotonic saline to remove the isopropanol. Since the device serves as a graft rather than as a wound dressing, it is cut to fit the wound shape and sutured or stapled in place on the excised would bed with the collagen-GAG sponge in contact with the wound bed.²

### Artificial Skin Quality Control Assays

The most important quality control assays for these experiments are: Average pore size, which can be measured by stereology⁴⁰ applied to scanning electron micrographs of a cut edge of the sponge; Endotoxin measured by commercial assay kit; and Peel strength between silicone and collagen-GAG layers.

### Electrospun Microfibers

Fibers of diameters 2.0 to 27.0 μm have been prepared. To prepare polypropylene fibers, a vessel of polypropylene is heated to approximately 210° C. and then single fibers are drawn through a nozzle, a process that results in smooth, cylindrically shaped fibers varying in diameter depending on the draw rate.

### In Vivo Assays

A well understood guinea pig model that has been the basis of artificial skin development will be used initially. Guinea pig studies are carried out as described by Yannas et al.³⁴ A more detailed protocol follows.

### Guinea Pig Surgery and Necropsy

Hartley guinea pigs, one to two months of age, weighing 400-500 g each are randomly assigned to groups containing an appropriate number of animals per test article and housed in large cages, four to a cage. After surgery, they are housed in individual cages. Food and water are given ad lib. Food is commercial guinea pig formula, which is withdrawn the night before surgery. Guinea pigs are shaved and residual hair removed with a commercial depilatory (Nair®) the previous day or the morning of a study. The hair is removed from the entire back and halfway down the sides. Tetracycline is given subcutaneously at a dose rate of 0.1 mg/kg. The guinea pigs are anesthetized using halothane at a concentration of 2.5%.

The animal's back is prepped with Betadine and then the animal is laid on sterile surgical towels. The guinea pigs are now ready for surgery. The recipient site is marked with Mercurochrome to the size of the graft, about 1.5×1.5 cm, and then prepped with 70% isopropanol and draped. The graft is placed on the mid portion of the back, slightly to the left of the spine. For paired comparative experiments, two grafts are placed, on either side of the midline. An incision using a #10 surgical blade is made around the perimeter of the marked 1.5×1.5 cm2 graft area down to the panniculus carnosus. One corner of the skin is picked up with forceps. Keeping tension on the corner, the surgical blade is used to excise the area down to the panniculus carnosus without cutting into it. After excision, the site is covered with a sterile dressing sponge to stop any bleeding. The artificial skin is placed in the recipient site and sutured in place using 5-0 Ethicon suture. Ten sutures are placed in each graft. Neosporin ointment is used along the wound edge to decrease the chance of any infection. The graft is covered with a sterile sponge and two wraps of Elastoplast. The guinea pigs are placed in a warm environment to recover from anesthesia. To compare the effect of the biomaterial matrix graft to open control wounds, the same surgical procedure is performed except the biomaterial matrix graft is not sutured into the wound. The open wound is bandaged as with the grafted sites and allowed to heal. The grafts and wounds are examined at periodic intervals and rebandaged if necessary until the animals are terminated.

At intervals following surgery, the animals are sacrificed and the graft site including 2-5 mm of normal surrounding tissue is removed. The tissue specimen is placed in 10% formalin, processed and stained with hematoxylineosin (H&E) for histological evaluation. Our technical expertise includes all of the basic skills for processing, embedding, and sectioning fresh specimens, as well as soft and hard tissue specimens that have been embedded in paraffin, plastic, or acrylamide.

H&E staining has historically been used in the development and optimization of the Integra Artificial skin and has demonstrated itself to be adequate for the development of an FDA approved and marketed product. This experience relieves us of the necessity of methods or model development and validation during Phase I research and allows us to concentrate our efforts on our engineering goals. However, immunohistological staining is available to us and will be used to supplement H&E, when appropriate.

### Histology

### Foreign Body Giant Cells (FBGC)

In observations of FBGC, there is an important distinction between those with identifiable foreign matter (starch granules, cotton, hair, suture, etc.) and substances associated with the collagen-GAG matrix in the absence of recognizable foreign matter. FBGC associated with foreign particles in the interface between matrix and host tissue bed generally indicate foreign matter that unavoidably contaminates the wound during the surgical procedure. When found within the matrix, it may indicate contamination of the matrix during its manufacture. FBGC associated with the matrix fibers in the absence of recognizable foreign matter are also occasionally observed. Large numbers of them may indicate an insufficiently biocompatible matrix. Neutrophilic Infiltration: Typically, few neutrophils are seen in the matrix during healing. Heavy infiltrations or other signs of infection may exclude a sample from further analysis.

### Other Cellular Responses

By day 7, the lower collagen-GAG matrix should show ingrowth of buds and tufts of mesenchymal cells with oval to spindle configurations. Small endothelial cell lined vascular spaces may be found at the base of these tufts. By day 10, typical fibroblasts are recognized. From day 10 on, the mesenchymal tissue proliferates and differentiates into moderately vascular fibroblastic connective tissue with birefringent collagen fibers until lattice spaces are filled by about day 22.

### Inflammation and Immune Reactions

Typically, few neutrophils are seen in the matrix during healing. Heavy infiltrations or other signs of infection exclude a sample from further analysis. However, the 10- to 35-day period is characterized by an advancing growth of epidermis and connective tissue over the collagen-GAG matrix from the wound edge below the silicone. When the mechanical silicone protective covering becomes weakened and lost (unmodified artificial skin), there is a local risk of acute inflammation. Otherwise, there should be no acute inflammatory response to the collagen-GAG matrix material alone. The presence of lymphocytes in the collagen-GAG matrix should be consistent over the healing period. The infiltrations range from trace to mild and are always diffuse; they are not in aggregates and never in lymphoid follicles. Plasma cells are not seen. Eosinophils are usually not seen in the collagen-GAG matrix before day 20. After 20 days, eosinophils may be present in trace to moderate numbers in the matrix as well as in normal skin adjacent to the collagen-GAG graft, in the normal skin of control animals, and in the healed open wound scars. Thus the presence of eosinophils alone does not appear to indicate an allergic response.

### Scoring

H&E stained slides are coded and blindly scored by one or more experienced observers according to a visual analog scale, with the endpoints of the scale being labeled 0 and 10 (Table 3). For all of the scoring markers, higher scores represent “better” performance, based on our understanding of parameters that may contribute to better in vivo performance. Thus a score of 10 is used for few FBGC, for a “wavy” non-oriented cell pattern, for a high cell density for few “myofibroblasts,” and for a high “tide mark.” Wounds showing pus, infection, or substantial hematoma are not scored. For scorable slides, evaluations are made in the middle third of the wound, since the edges of the wound are more complex, due to ingrowth of tissue from the margins as well as from the wound bed. Areas affected by small hematomas are ignored.

TABLE 3
Scoring of Guinea Pig Histology

| | Visual Analog Scale | | |
|---|---|---|---|
| Observation* | 0 | midpoint | 10 |
| Foreign Body Giant Cells | many | moderate | very few |
| (FBGC) in matrix | | | |
| FBGC associated with | many | moderate | Very few |
| biomaterial fibers | | | |
| Collagen + cellular fibrous | parallel | intermediate | wavy |
| pattern | oriented | | |
| Density of cells + matrix | Very few cells, | some open | fully packed |
| material | mostly open | spaces | with cells |
| | space | | |
| Activated fibroblast | many, parallel | intermediate | Very |
| phenotype | oriented | | few/wavy |
| | | | pattern |

*Other salient histological observations are made but not scored because they do not generally reflect differences in device performance.
† Foreign Body Giant Cells (FBGC): FBGC associated with the matrix fibers in the absence of recognizable foreign matter are occasionally observed in control artificial skin. Large numbers could indicate contamination during manufacture. FBGC associated with foreign particles in the interface between matrix and host tissue bed generally indicate foreign matter that unavoidably contaminates the wound during the surgical procedure, and should not be used to score the implant.

### Statistical Methods

Statistical analysis of initial guinea pig experiments will be based on ANOVA of the differences between treatment and control visual analogy scores (or from image processing measurement) from the paired wound sites. This design will give us ability to detect differences in performance of the artificial skin with and without fibers or of fiber performance between artificial skin and open wound environments.

If we are satisfied that we have met our objectives, we can switch from paired comparison with control to a balanced blocked design in which the test articles are applied to both wound sites on the animal. Controls consisting of unmodified artificial skin (without fibers) or fibers in open wounds will be included as treatment groups. This design doubles the number of wound sites available for test articles and will increase the power for detecting differences between test articles in searching for an optimum.

### Example 2

### Prosthetic Need

At present there are many treatment options for loss of a limbs or parts of limbs, including revision amputation, replantation, open treatment, prostheses of various forms (for fingers, hands/feet, arms/legs: myoelectric, shoulder-powered, cineplasty), or most recently, transplantation⁴¹. None of these succeed in restoring the lost limb or body part with normally functional tissue derived from the person sustaining the injury. Prosthetic replacement is still the most common option for most limb loss.

For any prosthesis to function, it must interface with the residual limb to adequately transfer the loads of physical support, motion and control. This is traditionally achieved through an intimately fit socket. The socket is shaped to contain the volume of the residual limb segment while distributing interface stresses in a manner tolerated by the tissues. Practically, this balance of loads in the dynamic situation of the prosthesis is extremely difficult to optimize and the result is often socket induced pain and reduced function, even in cases considered to be quite successful from the standpoint of conventional prosthetics.

While many amputees function with the traditional socket style prosthesis, this system has many inadequacies. Pressure points result in skin breakdown and discomfort. Socket pain results in the inability to wear the prosthetic device over long periods of time. Skin reaction and breakdown is a frequent problem with amputees requiring time out of the prosthetic device, treatment for infections, ulcerations, or even surgery. Many patients simply cannot tolerate socket style prosthetic devices. The most important clinical advance offered by the LET prosthesis is the elimination of the prosthetic socket, hence the elimination of the majority of the common problems of the prosthesis user.

The other limitation of the current system is the bone/implant interface. While the bone/implant interface has advanced further than the skin/implant interface⁴², it is has still not provided the durable long-lasting integration that will be needed for true success^{23,24,43,44,45,46,47}. The second major goal for this proposal will be to use advanced composite material designs that will improve this bone/implant interface resulting in a more permanent solution.

The proposed research will use new tissue-engineering technologies which promise to address the problems seen in clinical practice with transcutaneous prosthetics. We will also follow the chemistry and kinetics of bone formation around orthopedic implants⁴⁸. The specific aims of this proposal—advancements in cutaneous/implant interface and bone/implant interface—would have tremendous applicability across many medical disciplines.

### Composite Materials

Advanced composites are highly versatile and can be engineered for specific structural and morphological applications. Composites are multiphase (usually two) materials comprises a stiff, strong, oriented reinforcing phase embedded in a relatively soft, weak matrix phase. Well-established compositing and laminating techniques permit the designer to tailor the inherently anisotropic response of composites to achieve the optimal structure that satisfies the directionally sensitive stiffness and strength requirements of a particular application. The mechanical response of the composite is determined primarily by the type of fiber, fiber length (discontinuous or continuous), fiber architecture (direction and volume fraction), and fiber/matrix microstructure. Not only can the structural response of composites be tailored but the material characteristics such as porosity, morphology (through fiber selection, architecture, and processing), and interstitial characteristics can also be designed (within limits). This ability to locally tailor the properties of a composite pylon provides the opportunity to design an integral interface for the artificial skin scaffolding system while also providing the structural response necessary to transmit the induced loads to the host bone. The geometry as well as the compliance of the composite can be varied such that the pylon can closely mimic the loading due to the natural bone. Because composite materials can be made from a large variety of fibers and matrices, the requirement of biocompatibility can be accommodated by selecting constituent fibers and matrix materials which are biocompatible. It is proposed to use a carbon fiber reinforced thermoplastic resin, polyetheretherketone⁴⁹ (carbon/PEEK), as the LET Prosthesis composite structural material. Carbon/PEEK is lightweight (˜37% of titanium) and has demonstrated excellent biocompatibility^{19,21,50,51,52,53,54,55}, is FDA approved and is currently being used in long term human implants^{55,56,57}. The use of a composite structural member may not only eliminate the resorption of the host bone^{58,59,60,61,62,63}, but will also provide biofidelic response at the skin/prosthesis interface: i.e. it will have a mechanical response or compliance which will not overly strain the skin-prosthesis interface. The mechanical response, strength, and compliance, will be validated through mechanical testing as well as animal trials.

### Reinforcing Fibers

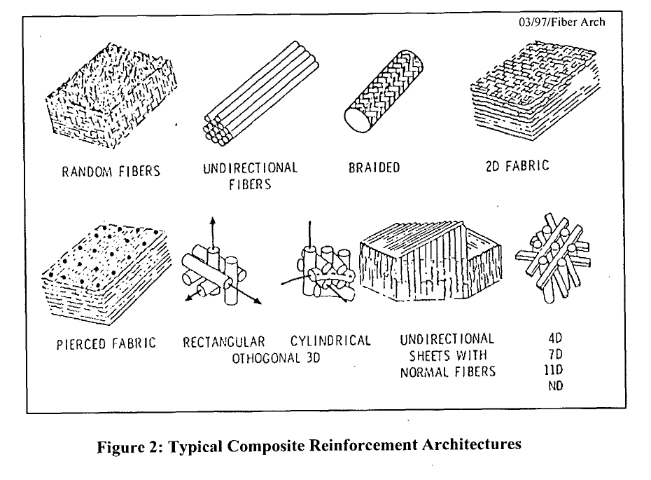

The anisotropic nature of continuous carbon fiber composites results in part from the fact that the carbon filament properties are themselves highly anisotropic, having a high longitudinal elastic modulus with a ratio of transverse to longitudinal modulus on the order of 0.1. In conventional carbon composite designs, one finds that strength and elastic modulus are proportional to the directional volume fraction of the filaments. Hence, carbon fiber directions or architecture are tailored to provide the directional strength and modulus required. Composite parts may be developed with a large variety of fiber architectures as illustrated in FIG. 2. The number of fiber directions which may be employed is very large and either straight or curved filament bundles may be used. In general, the larger the number of fiber directions, the lower the directional fiber volume fraction and the lower the directional strength of the composite. On the other hand, the larger the number of fiber directions, the more isotropic the composite properties.

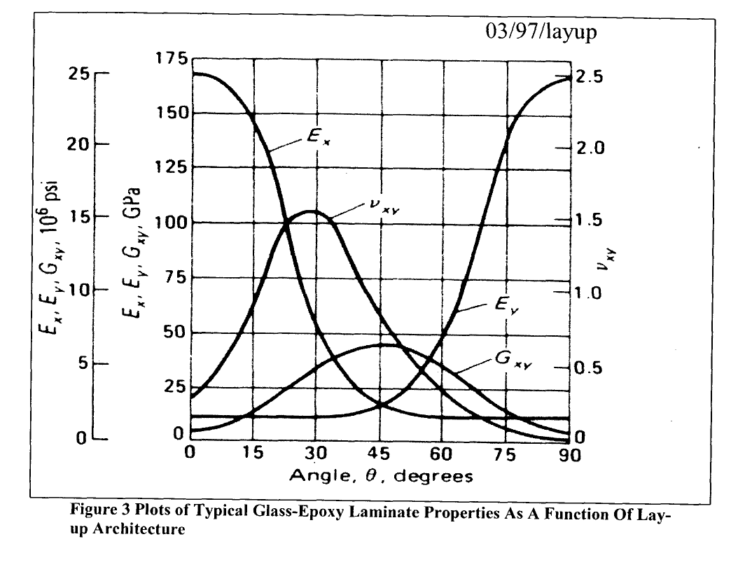

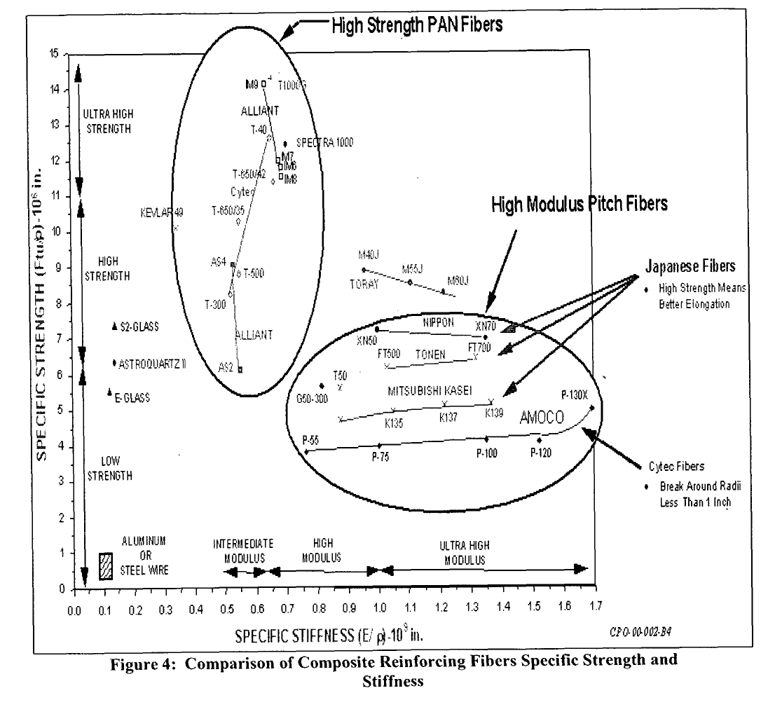

Also illustrated in FIG. 2 are several discontinuous carbon fiber architectures including carbon felt forms which usually result in fairly high porosity, relatively low-strength composites. FIG. 3 shows the typical property variation that can occur due to material lay-up geometry⁶⁴. FIG. 4 is a comparison of specific stiffness and strengths for most advanced reinforcing fibers. Ultra-high modulus carbon fibers are usually pitch based but have lower tensile strength than PAN. Not all fibers have application to the current program because of factors other than strength or stiffness. Ceramic (Nextel 440), metal (Boron), Glass (S2), Kevlar, and high temperature fibers (Astro Quartz II) are either too brittle, damage intolerant, expensive, or not biocompatible. Some graphite fibers (P100 and similar stiffness and strength fibers) are high stiffness/high cost fibers and would not provide the most effective design. The remaining carbon fibers fall into two broad bands of increasing specific stiffness and increasing specific strength. Ten fold, or greater, increases in specific stiffness relative to steel is achieved in a unidirectional (UD) lay-up with 60% reinforcement of the relatively new, high stiffness, pitch carbon fibers such as Cytec's P120 and P130. Similarly a 60% UD lay-up of Toray 1000G carbon fiber will produce a five-fold increase in strength. Even in pseudo-isotropic lay-ups of the same reinforcement volume fraction, specific stiffness increases of two to five, relative to steel alloys, are achievable. Compressive strength of the composite cannot always be directly related to the fiber strength, but also depends on the fiber bending stiffness, matrix modulus, and fiber/matrix interface properties. For example, pitch based composites usually have relatively low compressive strengths because of a poor fiber/matrix interface. And even though boron fibers have a relatively low specific tensile strength, composites reinforced with boron fibers have excellent compressive strengths (e.g. B/Epoxy and B/A1). This is due to the excellent bending stiffness of boron fibers. Another consideration is that most fibers have a sizing (a thin polymer coating), which is placed on the fibers by the manufacture to facilitate a good bond between the matrix and fiber. However, in many cases, this sizing is for a specific type of polymer matrix and may not be compatible with a different polymer. The composite designer must account for this in the constituent fiber/matrix selection.

### Matrix Materials

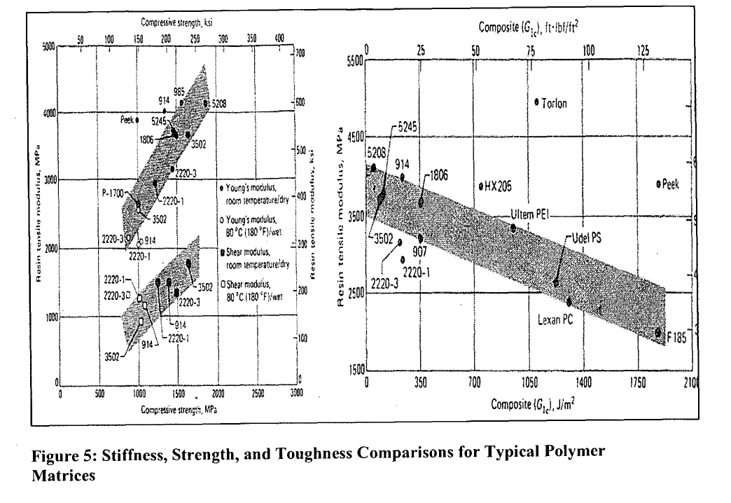

Important properties for the matrix resins include toughness, stiffness, strength, and ductility, response to various sterilizing methods, and biocompatibility for implantable devices. It is not only necessary to have good toughness, but the matrix must be stiff enough to transfer the load to the fibers. The many resins available for composites fall into two broad classes: thermoplastics (TP), which reversibly soften and melt with increasing temperature; and thermosets (TS), which irreversibly char and oxidize as temperature increases. However, all resin matrices have relatively low elastic moduli (on the order of 0.5 Msi) and low tensile strength (in the 10 to 20 ksi range). Therefore, matrix selection for orthopedic and prosthetic applications is based on other criteria; i.e., biocompatibility, environmental sensitivity (sterilization and in-use); impact resistance; damage tolerance and fracture toughness; ductility; and fiber/matrix compatibility. FIG. 5 gives strength, modulus and toughness data comparisons for typical thermoset and thermoplastic resin matrix systems^{65,66}.

### Prosthetics & Orthopedics Research Design and Methods

### Introduction

It is proposed to design the composite pylon in two sections: 1) proximal pylon which is osseointegrated with the host bone and 2) the distal pylon which incorporates the bacterial barrier. The two sections would be connected with a suitable structural connection such as a trunion or threaded joint. Although the LET Prosthesis is intended to be permanent, the modular design would allow a relatively easy replacement, if necessary, of a damaged distal component without having to remove the osseointegrated proximal component. This would also facilitate the ability to convert to a socket design if the LET Prosthesis was not a suitable system for the patient, i.e.: modular components which would allow the transcutaneous (distal) component to be removed and closing the wound to accommodate a standard socket system or, in the worst case, to be able to take out the osseointegrated (proximal) implant (composites can be easily drilled) to revert to a “socket” prosthesis.

The ability to tailor composite materials is an enabling technology that allows an optimization of the pylon-skin interface as well as the osseointegration of the pylon with the surviving bone. As shown in FIG. 6, the ambulatory loading of the in vivo pylon will likely be proximal to the bacterial barrier region to prevent high loads in the skin interface region. In addition, the proposed baseline skin interface is proposed to be proximal to the distal end of the LET prosthesis to prevent accidental loading of the skin or skin/implant when the external prosthesis is not present.

### Requirements Definition

We propose to use a lower limb amputee as a baseline initial clinical application to develop the load requirements. The use of a lower limb amputee clinical condition will allow us to use the data that is being generated on the current research project for lower limb amputees⁶⁷. Data from this current research will be used to develop the loads on the composite pylon and the residual bone. The load data will be used to design the composite transcutaneous pylon structural interface as well as the host bone/pylon load transfer, geometric constraints, and the external prosthesis interface.

Initial requirements for the transcutaneous pylon are that it be biocompatible, provide structural support to transfer load from the ground to the skeletal member, load the bone as close as possible to the normal physiological loading of an intact limb and provide a soft tissue bacterial barrier interface with the patient's skin.

### Composite Pylon-Osseintegration Development

The primary focus will be to develop a design, which will load the patient's bone in a near normal manner. We propose to accomplish this by designing the composite material to have a compliance close to that of the host bone and transfer the loads to the cortical and cancellous bone in a near normal physiological manner. Composites offer the ability to directionally vary the material modulus by fiber selection and architecture. Geometric features can also be molded into the composite to provide a biofidelic load path. We propose to use bone in-growth to provide a high strength mechanical interface and efficacious load transfer between the composite pylon and the host bone. As discussed above, the surface porosity of polymer composites can be designed to accommodate vascular tissue growth. Pore sizes of at least 75 microns are needed for osteon penetration. The optimum pore size for bone in-growth is approximately 100 to 350 microns⁶⁸. This type of surface porosity can be achieved by several different methods or combinations of methods. One method is treating the mold surface, in the case of net molding, to create a suitably rough surface using processes such as electrical discharge machining (EDM), chemical etching, and plasma sprayed metal powder, etc. This roughness is transferred directly to the composite part during fabrication. We are currently using this method to achieve a specific surface roughness on our spinal implants. The mold can also have ridges, “spikes” or dimples to obtain large/controlled pore sizes and penetration depth. Another method for achieving deeper pore penetration is to use “washout” matrix material in a local area. In this method, the washout matrix is a material that can be removed with a solvent or heat after the structure is fabricated. Coarse or fine weave fiber architecture can also achieve varying local surface characteristics

It is assumed that carbon/PEEK is the structural composite material, however, there are many candidate carbon fibers and several candidate PEEK type polymers which could be used.

### Composite Pylon Structural Design

Earlier studies in osseointegration have shown the value of detailed finite element analysis (FEA) on predicting the clinical results^{69,70,71}. While most of these earlier studies were conducted using porous metal components, they show the implant geometry and compliance are important factors in the performance and that the successful analytical requires a sophisticated, detailed modeling approach as described below. Earlier analytical work has demonstrated that direct skeletal attachment efficacy is related to the medullary geometry, cross section and length, as well as the external loading on the cortical bone through a collar type design⁷².

The global composite properties are generated based on the constituents, ply properties, and lay-up pattern. A detailed 3D finite element analysis (FEA) is performed for the component subjected to the prescribed mechanical loads and boundary conditions. For conventional metals, the next step is to compare the global stresses and strains with the material allowables. However, for composites the failure prediction is more complex.

Because both first-fiber and first-ply failure may occur in the same material, it is imperative to resolve the deformations/strains in the principal directions relative to each composite layer.

The pylon must be able to take the full structural loading imposed by ambulation. The geometric and load path design will be developed to load the cortical and the trabecular bone to closely simulate the natural physiological load path. For the purposes of this proposal, it is assumed that the proximal LET Prosthesis will incorporate a “collar” for loading the cortical bone as well as a medullary canal stem (see FIG. 6). The pylon design will be developed using both closed form and 3-D finite element analyses (linear, non-linear, and contact surfaces). The FEA will incorporate the bone, cortical and cancellous, properties, and bacterial barrier properties, as well as the loading and geometry requirements discussed above. The pylon fiber architecture and pylon geometry will be developed based on the FEA results. While the overall pylon composite analysis and design will focus on the specific requirements imposed by the bacterial barrier design and the osseointegration requirements as well as the proximal/distal pylon interface connection.

### Composite Pylon Bacterial Barrier and Bone Interface Fabrication

A high degree of precision in both geometric tolerance and composite architecture will be required. It is also assumed that the bacterial barrier and the osseointegration interface will require special surface characteristics such as controlled porosity and precision geometry. Because of this, it is assumed that state of the art net molding will be one of the primary composite fabrication technologies used in this proposed research. Net molding or near net molding is a closed mold fabrication methodology that uses hard tooling to control the part geometry. Each mold segment consists of a combination of rigid and/or semi-rigid sections that detail the features of the given part. The molds can be stand-alone components, which can be microprocessor controlled, be integrally heated, and capable of producing their own consolidation/compaction forces. This minimizes or eliminates the need for additional support equipment such as autoclaves and ovens etc. The integral heaters can be zoned in such a way as to give the controller precise control over the heat up rate of each zone. By preferential heating the interior mold zones of the tool and using the coefficient of thermal expansion (CTE) differential between the rigid and/or the semi-rigid tooling materials, the mold can create the necessary compaction forces to consolidate the laminate. Compaction is also obtained by using internal or external actuators, which can also be heated. Surface porosity, if required, can be achieved several ways depending on the required porosity and strength. In addition, metal or composite inserts can be molded in to achieve specific local requirements such as increased shear strength or surface texture.

Other fabrication options include fiber reinforced injection molding, tape wrapping, fiber placement, pultrusion, RTM (resin transfer molding), and/or standard vacuum bag processing.

### Transcutaneous and Osseointegration Animal Trials

### Osseointegration Animal Trials

A total of twelve rabbits will be studied, six each for three and six month follow-up of bone/implant healing. A minimum of four additional rabbits will be used as controls in each phase. The control rabbits will incorporate a titanium rod, which simulates (material and porosity) and osseointegration stems. Two of these rabbits will be sacrificed at six months and two at nine months.

A two-part composite pylon with a circular cross section with porosity of approximately 75 to 400 microns. The detailed design criteria from FE analysis will determine the optimum intramedullary segmental geometry and length. It is proposed that the proximal part of the two-part implant and the distal part of the two-part implant will be of different design criteria in order to test two different designs. The implant will be designed and fabricated in two parts to facilitate implantation into the animals.

The proposed pylon used for small animal testing will be an intercallary femoral section of ⅓ the length of the rabbit femur. A lateral approach to the femur will be made, the tensor fascia divided longitudinally, and the vastus lateralis will be elevated. The central third of the femur will be excised. Both ends of the two-part intercallary implant will be sized and shaped from the FE analysis model.

The intramedullary portion of the rabbit femur will be reamed and prepared. Part one of the implant will be placed proximally and part two distally. The two sections will be joined by a mechanical lock or by a biocompatible adhesive bond to restore femoral structural stability. The vastus lateralis fascia will be closed, and the tensor fascia repaired prior to skin closure. Post-operatively, the operated limb will be bound in flexion for four weeks to keep the animal from weight bearing. The animals will then be allowed to weigh bear as tolerated, and will be observed to determine when gait resumes and limping ceases.

Three animals will be harvested at month three, six, nine and twelve in order to examine the bone implant interface at both the proximal and distal ends of the two-part implant to determine the percent of the porous surface covered with bone in growth (osseointegration), versus fibrous in growth. Comparison of three, six, nine and twelve month specimens will also be performed to gauge the maturation process, and evolution of initial in growth over the first year. It is also proposed to perform mechanical push-out tests to a measure of the strength of the osseointegrated joint. Specimens from one of the rabbits with the titanium implant and one with each of the composite implants that was sacrificed at six and nine months will used in mechanical “push out” test as comparison between the “standard” titanium implant and the composite osseointegration implant. The other control rabbit implant will be sectioned and compared to the in-growth characteristics of the composite implants.

The information gathered in will be used to refine the FEA model, and produce two new iterative designs of porosity and composite structure. Twelve more rabbits will undergo implantation of the new two-part intercallary femoral implant. The surgical procedure, post-operative course and schedule for sacrifice of the animals is planned to be identical to the first trial, unless changes are mandated from information gathered in the first cycle.

### Proof-of-Concept Large Animal Trials

A dog model with a hindlimb amputation. 20 to 40 kg mongrel dogs will be selected for this hindlimb amputation model. Hindlimb amputations will be performed using standard amputation techniques to handle the skin flap design, bone, nerve, and muscle tissues. The flaps will be designed to allow percutaneous penetration of the implant, avoiding the site of the surgical incision.

Unlike the current osseointegrated implant, which requires a staged procedure where the bone implant is applied and three months later the percutaneous pylon is introduced into the bone implant, our plan is for a one-stage surgery, where the osseointegrated bone implant is implanted. The pylon and the cutaneous implant are also attached during this first surgery. The artificial limb will not be applied until the osseoimplant interface and the cutaneous/implant interface have matured. This timeline will be directed from the small animal trials. Animals will be allowed to function and walk on the prosthetic limb for six months and observed closely. If the animals fail to load the percutaneous prosthetic device normally in their gait pattern, the other hindlimb will be casted in knee flexion to render gait impossible without using the prosthetic limb device.

Animals will be killed six months after full weight bearing was initiated to retrieve the device and perform histologic evaluation of the osseoimplant interface and cutaneous/implant interfaces. We will section the bone/implant construct to evaluate the interface for fibrous tissue, direct bone integration, samples of local tissue for evidence of particular debris, and evaluation of the cellular response. In the event that no composite succeeds at osseointegration and the backup titanium bone component is used, no sectioning of the bone/implant interface will be possible, and instead evaluation of tissue removed from the implant surface will be performed.

Having now generally described the embodiments, the same may be more readily understood through the following reference to the following example, which is provided by way of illustration and is not intended to limit the present invention unless specified.

### REFERENCES

1. Yannas, I. V., J. F. Burke (1980). Design of an artificial skin. I. Basic design principles. J. Biomed Mater Res 14, 65-81.
2. Burke, J. F., Yannas, I. V., Quinby, W. C. Jr, Bondoc, C. C., Jung, W. K. (1981). Successful use of a physiologically acceptable artificial skin in the treatment of extensive burn injury. Ann Surg 194,413-428.
3. Heimbach, D., Luterman, A., Burke, J., Cram, A., Herndon, D., Hunt, J., Jordan, M., McManus, W., Solem, L., Warden, G., Zawacki, B. (1988). Artificial dermis for major burns. A multi-center randomized clinical trial. Ann Surg. 208, 313-320.
4. Heimbach D M, Warden G D, Luterman A, Jordan M H, Ozobia N, Ryan C M, Voigt D W, Hickerson W L, Saffle J R, DeClement F A, Sheridan R L, Dimick A R. Multicenter postapproval clinical trial of integra(r) dermal regeneration template for burn treatment. J Burn Care Rehabil 2003 24:42-8.
5. Larson J P, Congenital Amputation. In: Gale Encyclopedia of Medicine (online 2001).
6. Branemark R, Branemark P I, Rydevik B, Myers R R; “Osseointegration in skeletal reconstruction and rehabilitation: a review”; J Rehabil Res Dev. 2001 March-April; 38(2):175-81.
7. Bill Contoyannis; Rehab Tech, Monash University; personal communication, January, 2002.
8. Jacobs R, Branemark R, Olmarker K, Rydevik B, Van Steenberghe D, Branemark P I.; Department of Periodontology, Faculty of Medicine, Catholic University of Leuven, Belgium.; “Evaluation of the psychophysical detection threshold level for vibrotactile and pressure stimulation of prosthetic limbs using bone anchorage or soft tissue support.”; Prosthet Orthot Int 2000 August; 24(2):133-42.
9. Williams W G, Phillips L G, (1996). Pathophysiology of the burn wound. In Total Burn Care, (D. N. Hernndon, ed). 63-69.
10. Schulz J T 3rd, Tompkins R G, Burke J F, (2000). Artificial skin. Annu Rev Med 51:231-44.
11. Hiley, P and Barber, P C. General Pathology (Pathology Foundation Course), Chapter 3, Healing and Repair. Department of Pathology, University of Birmingham, U.K. http://medweb.bham.ac.uk/http/depts/path/Teaching/foundat/repair/healing.html.
12. W. A. N. Dorland, Dorland's illustrated medical dictionary. 29th Ed., W.B. Saunders Company, Philadelphia (2000).
13. F. Cahn, “Modification of natural polymers: collagen-glycosaminoglycan copolymers” in Methods of Tissue Engineering (Eds. A Atala and R P Lanza, Academic Press, New York), p 515-523, 2001.
14. Stem R, McPherson M, Longaker M T. Histologic study of artificial skin used in the treatment of full-thickness thermal injury. J Burn Care Rehabil. 1990 January-February; 11(1):7-13.
15. Moiemen N S, Staiano J J, Ojeh N O, Thway Y, Frame J D. Reconstructive surgery with a dermal regeneration template: clinical and histologic study. Plast Reconstr Surg; 108:93-103 (2001).
16. Standard Guide for Classification of Therapeutic Skin Substitutes. ASTM Standard F2311-03.
17. Boyce S T, Kagan R J, Yakuboff K P, Meyer N A, Rieman M T, Greenhalgh D G, Warden. Cultured skin substitutes reduce donor skin harvesting for closure of excised, full-thickness burns. Ann Surg 2002; 235:269-79.
18. Mansbridge J, Liu K, Patch R, Symons K, Pinney E. Three-dimensional fibroblast culture implant for the treatment of diabetic foot ulcers: metabolic activity and therapeutic range. Tissue Eng. 1998; 4:403-14.
19. Noordenbos J, Dore C, Hansbrough J F. Safety and efficacy of TransCyte for the treatment of partial-thickness burns. J Burn Care Rehabil. 1999; 20:275-81.
20. Hefton J M, Madden M R, Finkelstein J L, Shires G T. Grafting of burn patients with allografts of cultured epidermal cells. Lancet. 1983; 2:428-30.
21. Davila J C, Lautsch E V, Palmer T E. Some physical factors affecting the acceptance of synthetic materials as tissue implants. Ann NY Acad Sci; 146:138-147 (1968).
22. Bernatchez S F, Parks P J, Gibbons D F. Interaction of macrophages with fibrous materials in vitro. Biomaterials; 17:2077-2086 (1996).
23. Clark R E, Boyd J C, Moran J F. New principles governing the tissue reactivity of prosthetic materials. J Surg Res; 16:510-522 (1974).
24. Jansen J A, von Recum A F, van der Waerden J P, De Groot K. Soft tissue response to different types of sintered metal fibreweb materials. Biomaterials; 13:959-968 (1992).
25. Sanders J E, Bale S D, and Neumann T: Tissue response to micro-fibers of different polymers: polyester, polyurethane, polylactic acid, and polyurethane. J Biomedical Materials Research 62: 222-227 (2002).
26. There are several PEEK derivatives such as PEKEKK (polyetheretherketone) which has a trade name of Ultra-PEK or PAEK (polyaryletherketone). These materials have also shown excellent biocompatibility and will be considered in the proposed research. Their structural mechanics are similar. The primary differences are processing parameters and availability.
27. L. M. Wenz, K. Merritt, S. A. Brown, A. Moet; “In Vitro biocompatibility of polyetheretherketone and polysulfone composites”; J Biomedical Materials Research, Vol 24, 207-215, 1990.
28. Peluso, G., et al; “Biological response to chopped carbon fiber reinforced PEEK”; J. Biomedical Materials Research; 26(2):133-46; February, 1992.
29. M. F. Sigot-Luizard; “Biological Evaluation of the OSTA-PEK (Carbon-PEKEKK) Composites Used in Spinal Surgery”; Rachis, J. Vertebral Pathology, Spinal Surgery, Vol 12, No. 1; May 2000.
30. Sparta web site: www.composites.sparta.com.
31. Integra-LS.com.
32. G. E. P. Box and N. R. Draper “Empirical model-building and response surfaces,” John Wiley & Sons, New York (1987).
33. J. H. Brauker; R. C. Johnson; L. A. Martinson, and R. S. Hill U.S. Pat. No. 5,741,330, 1998.
34. Yannas I V, Lee E, Orgill D P, Skrabut E M, Murphy G F. Synthesis and characterization of a model extracellular matrix that induces partial regeneration of adult mammalian skin. Proc Natl Acad Sci USA; 86:933-7 (1989).
35. Yannas, I. V. Tissue regeneration by use of collagen-glycosaminoglycan copolymers. Clin. Mater. 9:179-187 (1992).
36. Yannas, I. V., Gordon, P., Burke, J. F., and Huang, C. Multilayer membrane useful synthetic skin. U.S. Pat. No. 4,060,081 (1977).
37. Yannas, I. V., Burke, J. F., and Stasikelis, P. J. Method for preserving porosity in porous materials. U.S. Pat. No. 4,522,753 (1985).
38. Yannas, I. V., and Tobolsky, A. V. Crosslinking of gelatin by dehydration. Nature (London) 215:509-510 (1967).
39. Yannas, I. V. Collagen and gelatin in the solid state. Rev. Macromol. Chem. C7,49B-104B. (1972).
40. Dagalakis, N., Flink, J., Stasikelis, P., Burke, J. F., and Yannas, I. V. Design of an artificial skin. Part III. Control of pore structure. J. Biomed. Res. 14,511-528 (1980).
41. Jones J W et al. Successful hand transplantation: one year follow-up. N Engl J Med 2000; 343:468-73.
42. Ysander M, Branemark R, Olmarker K, Myers R R; “Intramedullary osseointegration: development of a rodent model and study of histology and neuropeptide changes around titanium implants”;. J Rehabil Res Dev 2001 March-April; 38(2): 183-90.
43. T. Albrekisson; “On Bone Biological Research Behind Osseointergration”; 2nd International Conference Advances in Osseointegration; Univ of Surrey, Guildford, Surrey, UK; 15th-18th, June 2000.
44. R. Branemark; “Osseointegrated Transfemoral Amputation Prostheses”; 2nd International Conference Advances in Osseointegration; Univ of Surrey, Guildford, Surrey, UK; 15th-18th, Jun. 2000.
45. Kevin E. Healy, “Challenges in Achieving Direct Skeletal Attachment”; National Institutes of Health, NICHHD; Medical Rehabilitation on the Move: Spotlight on Bioengineering; Jan. 4-5, 2001.
46. Rickard Branemark; “Bone Anchored Amputation Prostheses”; National Institutes of Health, NICHHD; Medical Rehabilitation on the Move: Spotlight on BioEngineering; Jan. 4-5, 2001.
47. Rolf Branemark, John Sullivan, Maggie Udden, Kingsley; “Osseointegration for Prosthesis Limb Attachment”; Symposium (SY26), The 10th World Congress of the International Society for Prosthetics & Orthotics (ISPO); Glasglow. UK; Jul. 1-6, 2001.
48. Kevin Healy; “Challenges in Achieving Direct Skeletal Attachment”; Departments of Bioengineering and Materials Science and Engineering, University of California at Berkeley; Berkeley, Calif.; Abstract announced May 2, 2001; Sponsored by National Institute of Child Health & Human Development, NIH.
49. There are several PEEK derivatives such as PEKEKK (polyetheretherketone) which has a trade name of Ultra-PEK or PAEK (polyaryletherketone). These materials have also shown excellent biocompatibility (see M. F. Sigot-Luizard below) and will be considered in the proposed research. Their structural mechanics are similar. The primary differences are processing parameters and availability.
50. Victrex; “PEEK Product Guide (Medical)”; PEEK-Optima LT Polymer; VictrexUSA, Inc.; 601 Willowbrook Lane, West Chester, Pa. 19382; (610) 696-3144.
51. R. Leaversuch; “Legal ruling opens door for PAEK in human implants”; Plasticope; September, 1999.
52. S. A. Brown, R. S. Hastings, J. J. Mason, A. Moet; “Characterization of short-fibre reinforced thermoplastics for fracture fixation devices” Biomaterials, pp 541-7, 1990.
53. J. W. Brantigan, A. D. Steffee; “A Carbon Fiber Implant to AID Interbody Lumbar Fusion” Spine, vol 18, No. 14, pp 2106-2117; Nov. 14, 1993.
54. John W. Brantigan, M D; Arthur D. Stefee, M D; Jack M. Geiger, PhD; “A Carbon Fiber Implant to Aid Interbody Lumbar Fusion, Mechanical Testing”; Spine, vol 16, no. 6; Nov. 6 Supplement, 1991.
55. M. F. Sigot-Luizard; “Biological Evaluation of the OSTA-PEK (Carbon-PEKEKK) Composites Used in Spinal Surgery”; Rachis, J. Vertebral Pathology, Spinal Surgery; Vol 12, No. 1; May 2000.
56. Press Release, “Scient'x selects Invibio PEEK-Optima polymer for interbody fusion cages”; http://www.invibio.com/pr002.html; Aug. 8, 2001.
57. DePuyAcroMed, Brantigan Cage System; For Lumbar Fusion indication.
58. J. D. Bronzino, The Biomedical Engineering Handbook; pg 605, CRC Press, IEEEPress; ISBN 0-8493-8346-3, 1995.
59. J. B. Park, R. S. Lakes; Biomaterials; Plenum Press, New York; pg 300-306; ISBN 0-306-43992-1; 1992.
60. J. Black; Biological Performance of Materials: Fundamentals of Biocompatibility; 2nd Edition; Marcel Dekker, New York; 1992; ISBN 0-8247-8439-1; pg 174-181.
61. Huttner, W; Fitzer, E; et al: “Torsional Strength of Carbon Fibre Reinforced Composites for the Application as Internal Bone Plates”, Carbon. Volume 28, No. 6-A, 1980.
62. Tayton, Keith et al; “The Use of Semi-Rigid Carbon-Fibre” Reinforced Plastic Plates for Fixation of Human Fractures”, The Journal of Bone and Joint Surgery, Volume 64-B No. 1, 1982 (British).
63. Mendes, David G.; et. al., “Carbon Fibre Reinforced Polysulfone Hip Implant Experimental Study on Dogs”, Proceeding of the 1st Annual Scientific Session of the Academy of Surgical Research; San Antonio, Tex.; Oct. 12-19, 1985.
64. White, M.; “Development of an Advanced Lightweight Composite Orthosis.” ASM International Aeromat '92: New Trends in Advanced Composites. Anaheim, Calif. 1992.
65. Composites, Engineering Materials Handbook; ASM International Vol. I, pg 223, 1988.
66. Composites, Engineering Materials Handbook; ASM International Vol. I, pg 99, 1988.
67. Advanced Biofidelic Lower Extremity (ABLE) Prosthesis Research”; Grant No. R01 HD38933-01; NIH, National Institute of Child Health and Human Development; Dr. Louis Quatranno; Project Period: Sep. 10, 2000-Sep. 31, 2004.
68. Joseph Bronzino, The Biomedical Engineering Handbook, pg 680-723, CRC Press, IEEE Press, 1995.
69. Krouskop T A, Trono R, Adamski A J The effects of pylon shape on bone-pylon interface performance in direct skeletal attachment. J Biomed Mater Res 1976 May; 10(3):345-69.
70. Rickard Branemark; “Bone Anchored Amputation Prostheses;” National Institutes of Health, NICHHD; Medical Rehabilitation on the Move: Spotlight on BioEngineering, Jan. 4-5, 2001.
71. Viceconti M, Muccini R, Bernakiewicz M, Baleani M, Cristofolini L.; Laboratorio di Tecnologia Medica, Istituti Ortopedici Rizzoli, via di Barbiano 1/10, 40136, Bologna, Italy; “Large-sliding contact elements accurately predict levels of bone-implant micromotion relevant to osseointegration.” J Biomech 2000 December; 33(12):1611-8.
72. Xu W, Crocombe A D, Hughes S C; “Finite element analysis of bone stress and strain around a distal osseointegrated implant for prosthetic limb attachment”. Inst Mech Eng [H] 2000; 214(6):595-602.

What is claimed is:

1. A wound closure system, comprising: a porous layer which comprises a collagen material; a substantially non-porous synthetic layer contacting the porous layer, the porous layer and substantially non-porous layer capable of providing wound closure; and a transcutaneous component contacting the porous layer and the substantially non-porous synthetic layer.

2. A wound closure system according to claim 1, wherein the porous layer and the substantially non-porous synthetic layer surrounds the transcutaneous component.

3. A wound closure system according to claim 1, wherein the non-porous synthetic layer comprises silicone, polyacrylate esters, polyurethane or combinations thereof.

4. A wound closure system according to claim 1, wherein the collagen material comprises collagen-glycosaminoglycan.

5. A wound closure system according to claim 1, wherein the transcutaneous component is a pylon containing a metal, polymer or fiber.

6. A wound closure system according to claim 5, wherein the porous layer comprises biodegradable collagen-glycosaminoglycan having an average pore size ranging from about 50 microns to about 200 microns and the non-porous synthetic layer comprises silicone and the pylon comprises titanium.

7. A wound closure system according to claim 5, wherein the pylon is capable of receiving a cannula, glucose sensor, electrode, chest tube, medical instrument, prosthesis, bone or combination thereof.

8. A wound closure system, comprising: a porous layer comprising a collagen material and a substantially non-porous synthetic layer contacting the porous layer, each layer capable of receiving a transcutaneous component, the porous layer and substantially non-porous layer capable of providing wound closure by allowing growth of neodermal tissue and an anchoring material disposed within the porous layer.

9. A wound closure system according to claim 8, wherein the anchoring material comprises an inner region and an outer region, the inner region being less flexible than the outer region.

10. A wound closure system according to claim 8, further comprising a transcutaneous component, wherein the transcutaneous component is a pylon which contacts the substantially non-porous synthetic layer and the porous layer.

11. A wound closure system according to claim 8, wherein the anchoring material comprises polytetrafluoroethylene, polypropylene, polyolefin, gortex, or polyester fiber or combinations thereof.

12. A wound closure system according to claim 8, wherein the collagen material comprises collagen-glycosaminoglycan.

13. A wound closure system according to claim 10, wherein the pylon is capable of receiving a prosthesis at one end and bone at the other end.

14. A wound closure system according to claim 10, wherein the pylon is capable of receiving a cannula, glucose sensor, electrode, prosthesis, chest tube, medical instrument or bone or combination thereof.

15. A wound closure system according to claim 10, wherein the pylon comprises a metal or polymer.

16. A wound closure system according to claim 10, wherein the pylon comprises a proximal component that is capable of receiving bone and a distal component comprising a load bearing region that is capable of receiving a prosthetic device.

17. A wound closure system according to claim 10, wherein the porous layer comprises biodegradable collagen-glycosaminoglycan having an average pore size ranging from about 50 microns to about 200 microns and the non-porous synthetic layer comprises silicone and the pylon comprises titanium.

18. A wound closure system according to claim 8, wherein the porous layer comprises an antimicrobial agent.

19. A wound closure system according to claim 10, wherein the non-porous synthetic layer further comprises a permanent membrane contacting a region of the pylon.

20. A wound closure system according to claim 10, wherein a sleeve surrounds a region of the pylon.

21. A wound closure system according to claim 10, wherein the pylon is capable of receiving a cannula, glucose sensor, electrode, prosthesis, chest tube, medical instrument, bone or combination thereof.

22. A wound closure system, comprising: a porous layer and a substantially non-porous synthetic layer contacting the porous layer, the porous layer and the substantially non-porous synthetic layer capable of receiving a transcutaneous component and providing wound closure by allowing growth of neodermal tissue, the porous layer comprising biodegradable collagen-glycosaminoglycan, and a non-degradable anchoring material disposed within the porous layer.

23. A wound closure system, comprising: a porous layer which comprises a collagen material; a substantially non-porous synthetic layer contacting the porous layer, the substantially non-porous synthetic layer comprising removable silicon and a permanent membrane, the porous layer and substantially non-porous layer capable of providing wound closure; and a transcutaneous component contacting the porous layer and the substantially non-porous synthetic layer.

24. A wound closure kit, comprising: a porous layer comprising a collagen material capable of providing wound closure by allowing growth of neodermal tissue; and a substantially non-porous synthetic layer contacting the porous layer, the porous layer and substantially non-porous layer capable of receiving a pylon.

25. A method for providing wound closure surrounding a transcutaneous component, comprising: applying a wound closure system to a wound, the wound closure system comprising: a porous layer comprising a collagen material that allows growth of neodermal tissue; a substantially non-porous synthetic layer contacting the porous layer; and a transcutaneous component surrounded by the porous layer and the substantially non-porous synthetic layer.

26. A transcutaneous infection (foreign body) barrier system, comprising:
(a) a porous layer comprising a collagen material;
(b) a substantially non-porous synthetic layer contacting the porous layer, the porous layer and substantially non-porous layer capable of promoting wound closure; and
(c) a transcutaneous component contacting the porous layer and the substantially non-porous synthetic layer, the transcutaneous component having an integral subcomponent which allows physical incorporation of the porous and/or the non-porous layer into the transcutaneous component.

27. A transcutaneous infection (foreign body) barrier system according to claim 26, wherein an external load can be transferred through the patients' skin directly to the patient's skeletal bone.

28. A transcutaneous infection (foreign body) barrier system according to claim 26, wherein an infection barrier is established at the structural component/skin interface utilizing artificial skin integrally connected to the structural transcutaneous component to promote the patient's skin to form a permanent infection and/or foreign body barrier with the structural transcutaneous component.

29. A transcutaneous infection (foreign body) barrier system according to claim 26, wherein the transcutaneous component comprises two primary units, one unit capable of receiving an external prosthesis at one end and the other unit comprises the bone interface, the two primary units connected by an engineered structural joint capable transmitting load from an external source to an amputee's bone.
